(19) United States
(12) Patent Application Publication
(10) Pub. No.: US 2011/0008350 A1
(43) Pub. Date: Jan. 13, 2011

(54) EXTRACELLULAR TARGETS FOR ALZHEIMER'S DISEASE

(76) Inventors: Bart De Strooper, Leuven (BE); Tomoko Wakabayashi, Chiba (JP)

Correspondence Address: TRASKBRITT, P.C., P.O. BOX 2550, SALT LAKE CITY, UT, 84110, US

(21) Appl. No.: 12/733,993

(22) PCT Filed: Oct. 3, 2008
(86) PCT No.: PCT/EP2008/063269

§ 371 Date: Sep. 21, 2010

Related U.S. Application Data
(60) Provisional application No. 60/998,017, filed on Oct. 4, 2007.

(51) Int. Cl. A61K 39/395; A61K 31/7105; A61P 25/28

(52) U.S. Cl. 424/139.1; 514/44 A

(57) ABSTRACT

The present invention relates to the field of neurological disorders and more particularly to the field of Alzheimer's disease. Specifically, the invention provides extracellular targets for Alzheimer's disease selected from the tetraspanin web family and associated proteins. In addition, methods are provided for the use of siRNAs and antibodies against the targets for inhibition of amyloid-beta production and, hence, for the treatment of Alzheimer's disease.

### TECHNICAL FIELD

The present invention relates to the field of neurological disorders and, more particularly, to the field of Alzheimer's disease. Specifically, the invention provides extracellular targets for Alzheimer's disease selected from the tetraspanin web family and associated proteins. In addition, methods are provided for the use of siRNAs and antibodies against the targets for inhibition of amyloid-β production and, hence, for the treatment of Alzheimer's disease.

### BACKGROUND

Every cell biological process relies on transient or stable physical interactions of proteins, which, in association with lipids, sugars and other biomolecules, form complexes and functional networks in the cell. These interactions represent a pivotal aspect of protein function. Gamma-secretase, an aspartyl protease of the “intramembrane cleaving proteases (iCLiPs)” family, is a multiprotein complex responsible for the generation of β-amyloid peptides (Aβ), the primary component of the senile plaques in the brains of Alzheimer's disease (AD) patients. Apart from Amyloid Precursor Protein (APP), γ-secretase also cleaves a large number of type I membrane proteins involved in a wide variety of biological processes such as the Notch receptor, N-cadherin, ErbB-4 and syndecan-3.¹ The cell surface, endosomal and recycling compartments have been suggested as major places of γ-secretase activity.²The core of γ-secretase consists of four highly hydrophobic proteins: namely, Presenilin (PS), Nicastrin (NCT), Anterior pharynx defective-1 (Aph-1) and Presenilin enhancer-2 (Pen-2).³ Two different Presenilins (PS1 and PS2) were originally identified as products of major gene loci for early onset autosomal dominant AD. PS1 and PS2 provide the catalytic site, consisting of a pair of aspartates within two adjacent transmembrane domains, to the γ-secretase complex.^{4, 5} NCT is implicated in substrate recognition,⁶ whereas the functional roles of Aph-1 and Pen-2 have not yet been fully elucidated. These four proteins are the minimal components needed to form an active complex. For instance, in yeast cells, which do not express these proteins, γ-secretase activity was reconstituted only when the four components were expressed together.⁷ In mammalian and insect cells, these four components are indispensable and co-overexpression of them resulted in the increase of γ-secretase activity as measured by increased Aβ secretion.^{8, 9} However, the increment in activity was lower than expected from the protein expression levels obtained, which suggested that other factors are involved in regulation of γ-secretase activity. Indeed, the size of high-molecular-weight complexes having γ-secretase activity varies from ˜250 kDa to ˜2000 kDa, depending on the experimental conditions used, implying either oligomeric assemblies of the γ-secretase complex or associated proteins that transiently or stably interact with the core components.¹⁰⁻¹⁴ Several proteins have indeed been shown to interact with PS and/or the γ-secretase complex, such as β-catenin,¹⁵ Tmp21,¹⁶ CD147,¹⁷ and others.¹ However, a comprehensive approach to identify all proteins interacting and regulating γ-secretase activity has not been published until now. The identification of proteins regulating γ-secretase might also provide new molecular targets for the development of medication for AD. We have, therefore, put a tandem affinity-tag (TAP-tag) on PS and applied tandem affinity-tag purification (TAP) to map its interactome. TAP has been mainly used in yeast S. cerevisiae,^{18, 19} although recently also the interactome of TNF-alpha/NF-kappaB pathway was mapped in mammalian cells in this way.²⁰ The major strength of the TAP approach is its combination of two high-affinity binding steps and two highly specific elution steps, which results in efficient enrichment of interacting proteins.

By using this strategy, we were we were able to demonstrate for the first time that γ-secretase is partially associated with the tetraspanin web in the cell membrane. We have validated the proteins using RNAi experiments and fractionation studies. By doing so, it was demonstrated that three proteins derived from the tetraspanin web family are extracellular targets for Alzheimer's disease. Thus, molecules that bind on the targets can be used to treat AD.

### DESCRIPTION OF THE DRAWINGS

FIG. 1: Stable expression and TAP-tag purification of dTag PS and dTag SPPL3. (a) Stable expression of dTag PSs in PS dKO MEF cells. Cell lysates of MEF cells were analyzed by Western blot with antibodies against γ-secretase components and FLAG (M2). Expression of dTag PS1 or PS2 rescued the maturation of Nicastrin and stabilization of Pen-2. PS1 full length (fl), N-terminal fragment (NTF), mature (m) and immature (i) forms of Nicastrin are indicated. (b) Stable expression of dTag SPPL3 in PS dKO MEF cells. Western blot analysis indicates that dTag SPPL3 failed to rescue γ-secretase maturation. (c) Schematic representation of TAP-tag purification. (d) TAP-tag purification of dTag PS1. An equivalent amount of each fraction was analyzed by Western blot. γ-secretase components were quantitatively retained in the purified fractions (M2 and CaM eluate). CTF, C-terminal fragment. (e) In vitro γ-secretase assay of the purified fractions. Equivalent amounts of each fraction were mixed with recombinant substrate APP C99-3×FLAG, incubated at 37° C. and analyzed by Western blot with anti-Aβ antibody (W0-2). De novo generation of Aβ demonstrates the quantitative recovery of active γ-secretase. (f) TAP-purified dTag PS1 fraction (three-step) was separated on SDS-PAGE and stained with Coomassie blue. Arrowheads indicate predicted position of the γ-secretase components. Left lane, molecular standard.

FIG. 2: TAP-tag purification of dTag PS2, SPPL3 and PS dKO MEF cells. (a) TAP-tag purification of dTag PS2. Each fraction was analyzed by Western blot. γ-secretase components were retained in the purified fractions. (b) TAP-tag purification of dTag SPPL3 and Western blot analysis of each fraction. γ-secretase components remained in M2 flow through. (c) Control TAP-tag purification from PS dKO MEF cells.

FIG. 3: PS interacting proteins involved in the membrane trafficking and organization. (a) PS interacting proteins involved in membrane trafficking and membrane organization are displayed with the descriptions. (b) The proteins listed in Panel a are distributed in anterograde and retrograde pathways between the ER and Golgi, and endocytic/recycling pathways from/to the plasma membrane. PM, plasma membrane; EE, early endosome; LE, late endosome; LY, lysosome; ERAD, ER-associated degradation.

FIG. 4: PS interacting proteins and effects of siRNA-mediated knockdown on Aβ secretion. (a) Schematic representation of the tetraspanin web. The arrows between proteins indicate binding interactions that have been experimentally demonstrated in the literature^{35,36,50}. Dashed lines represent different levels of interactions. PS interacting proteins that are implicated in the formation of the tetraspanin web or are laterally associated with the tetraspanin web, are indicated. (b) RNAi-mediated knockdown effect of PS interacting proteins in HEK293 APPSw cells implicated in the tetraspanin web on Aβ secretion. Secreted Aβ40 and Aβ42 were measured by specific ELISA after 48 hours of transfection. Y-axis represents % compared to the Aβ levels of control (transfection with control siRNA pool). Data are presented as mean values and SEM of 6 tests. Significance was set at * P<0.05; ** P<0.01; and ***P<0.001. (c) siRNA-mediated knockdown of tetraspanin web proteins in HeLa cells. After 48 hours of transfection solubilized total cell lysate was analyzed by Western blot. Results shown in duplicate. (d) γ-secretase activity upon knockdown of tetraspanin web proteins in HeLa cells. HeLa cells were transfected with siRNAs as in panel c, followed by infection with recombinant adenovirus promoting expression of APPSw. Secreted Aβ40 and Aβ42 were measured by ELISA. Aβ levels are related to those in cells treated with control siRNA pool. Data are presented as mean values and SEM of three independent experiments. Significance was set at * P<0.05 and ***P<0.001.

FIG. 5: RNAi screening for γ-secretase modulators. (a) HEK293 APPSw cells were transfected with siRNAs targeting human orthologues of PS interacting proteins involved in membrane trafficking and others such as proteins with transporter activities and cell adhesion molecules. Secreted Aβ40 and Aβ42 were measured by specific ELISA after 48 hours of transfection. Y-axis represents Aβ levels in the RNAi treated cells compared to the Aβ levels in control cells (transfected with control siRNA pool). Data are presented as mean values and SEM of 4 tests. Significance was set at * P<0.05; ** P<0.01; and ***P<0.001. (b) Levels of full-length APP, APP CTF and γ-secretase components upon RNAi were analyzed by Western blot. Proteins whose knockdown significantly altered levels of either Aβ40, 42 or both were chosen for the analysis. Note that RNAi of VCP, Myadm and 4F2lc also affected levels of APP expression and APP-CTF. This suggests that these proteins affect APP processing not only at the level of γ-secretase but also at the level of APP trafficking.

FIG. 6: siRNA-mediated knockdown of p24 family proteins. (a) Western blot of HEK293 APPSw cells transfected with Tmp21 and p24a siRNAs. The cells were transfected siRNA twice and the cells were lysed after 56 hours of the second transfection. As previously indicated, knockdown of Tmp21 decreased the levels of p24a and vice versa. p24a RNAi affected APP levels. (b) Secreted Aβ was measured by ELISA. Knockdown of Tmp21 slightly augmented Aβ40, while p24a decreased both Aβ40 and Aβ42. Data are presented as mean values and SEM of three independent experiments. Significance was set at ***P<0.001.

FIG. 7: Overexpression of tetraspanin-related proteins. (a) Western blot analyses of HEK293 APPSw cells transiently expressed FPRP, PGRL, CD98hc (left panel), CD81-V5, Upk1b-V5 (middle panel) and CD9 (right panel). The cells were collected 36 hours after transfection. Expression of FPRP or PGRL augmented the levels of CD98hc. (b) Secreted Aβ was measured by ELISA. Data are presented as mean values and SEM of three independent experiments. Significance was set at *P<0.05 and ***P<0.001.

FIG. 8: Physiological interactions between γ-secretase and tetraspanin web-related proteins. (a) Confirmation of interactions between the γ-secretase complex and the tetraspanin web proteins by co-immunoprecipitation. 1% CHAPSO-solubilized HEK293 membranes were precipitated with anti-Aph-1a (B80.3) or preimmune serum (control) and the precipitates were analyzed by Western blot. Note that also tetraspanin CD9 (which was not identified in the MS experiments) is found to associate with the γ-secretase complex. (b) FPRP co-immunoprecipitated γ-secretase components. 1% CHAPSO-solubilized HEK293 membranes were incubated with anti-FPRP (1F11) or isotype control antibody and analyzed by Western blot. Stable overexpression of FPRP (c) and PGRL (d) in HEK293 cells. Immunoprecipitation of FPRP (1F11) and PGRL (8A12) from the stable cells showed increased association with γ-secretase components compared to wt cells. Asterisk indicates Ig bands. Expression levels of endogenous PGRL in HEK293 cells were below the detection level and PGRL was detected only after precipitation with anti-PGRL (d).

FIG. 9: Accumulation of CTF of γ-secretase substrates in CD81 and CD9 deficient MEFs. The levels of C-terminal fragments (CTF) of endogenous γ-secretase substrates in MEF cells deficient in CD81 or CD9 were analyzed by Western blot (left panel). Wt MEF cells treated with 10 μM γ-secretase inhibitor DAPT were used as a control (wt+inhibitor) to show accumulation of CTF. The levels of individual γ-secretase components or the substrates in these cells are unchanged (right panel).

FIG. 10: Codistribution of tetraspanin web proteins and γ-secretase components on sucrose density gradient. HEK293 cells were solubilized with 1% Triton X-100, 0.5% DDM, 1% CHAPSO or 1% Brij99 and separated on discontinuous sucrose density gradients. Thirteen fractions were collected from the top and analyzed by Western blot. In these experiments, membrane/lipid domains float to the top fractions when they remain associated. When detergents are used that disrupt the interactions, proteins get redistributed to the bottom fractions. Tetraspanin domains remain preserved in 1% CHAPSO and 1% Brij99 and γ-secretase components co-distribute with these domains in the gradient. Notice that also the caveolin-1 marker for rafts floats in those experiments as expected. The ER marker calnexin remains in the heavier fractions. Detergents like TX-100 or 0.5% DDM, which maintain rafts, are dissociating the tetraspanin domains and γ-secretase complex does not float any longer in the light fractions. The caveolin marker for rafts remains in the light fractions, indicating that raft domains remain indeed preserved in these conditions.

FIG. 11: γ-secretase associated with the tetraspanin web generates more long Aβ species. Proteolytic activity of γ-secretase associated with the tetraspanin web was measured by in vitro assay. (a) 1% CHAPSO-solubilized microsomal membranes from wt HEK293 cells and HEK293 expressing FPRP were subjected to immunoprecipitation with anti-FPRP, PS1, Aph-1a or control antibodies. Bound complex was incubated with recombinant substrates C99-3×FLAG at 37° C. for 3 hours and resulting AICD was analyzed by Western blot with anti-FLAG (M2) (upper panel). Specificity for the reaction was validated by a reaction of the membranes of wt cells incubated in the presence of γ-secretase inhibitor L-685,458 (input+inhibitor). Production of Aβ species was measured by urea-SDS PAGE and Western blot with anti-Aβ (82E1) (lower panel). Asterisks indicate nonspecific bands derived from the substrate. Synthetic Aβ peptides were used as molecular standards (Aβ std). (b) Aβ generation was quantified from the immunoblots of three independent experiments. Intensity of Aβ bands was measured and normalized to the sum of Aβ in each individual reaction. Data are presented as mean values and SEM. Significance was set at * P<0.05 and **P<0.01. (c) Immunoprecipitation with anti-CD81, CD9 and PS1 antibodies from 1% CHAPSO-solubilized microsomal membranes of MEF cells followed by in vitro assay. AICD and Aβ species were visualized by Western blot. Relative ratio of long Aβ species per total Aβ was measured and indicated (lower panel). (d) Aβ generation was quantified from the immunoblots of four independent experiments.

### DETAILED DESCRIPTION OF THE INVENTION

Gamma-secretase is a high-molecular-weight complex containing Presenilin, Nicastrin, Aph-1 and Pen-2 that cleaves type I membrane proteins. These four components are necessary and sufficient for γ-secretase activity, but additional proteins might interact. We purified, therefore, active γ-secretase complex from reconstituted Presenilin-deficient fibroblasts using a tandem affinity purification (TAP) approach. We identified proteins involved in complex maturation, membrane trafficking and the tetraspanin web family. The tetraspanin super-family of small, four transmembrane domain proteins (up to 350 amino acids) consists of 33 members in humans and mouse and includes proteins that are involved in physiological processes as diverse as egg-sperm fusion, immunological responses and tissue differentiation. According to topology predictions, tetraspanins have two extracellular domains (often referred to as the small extracellular loop and the large extracellular loop (LEL)) and three relatively short cytoplasmic regions. Previous experiments established that tetraspanins interact with one another and form a structural platform for the assembly of a novel class of microdomains (referred to as tetraspanin-enriched microdomains (TERM, TEM) or “tetraspanin webs”). It has been proposed that through a network of homotypic and heterotypic interactions, tetraspanins regulate the spatial juxtaposition of associated transmembrane receptors (e.g., integrins, receptor tyrosine kinases) on the plasma membrane, which results in coordination of signaling pathways.

There is also emerging evidence that tetraspanins regulate biosynthetic maturation and trafficking of their associated partners. In the present invention, we have identified three tetraspanin web family members that not only physiologically interact with the γ-secretase complex but also influence the production of amyloid beta. Thus, the three tetraspanin web family members (CD81, PTGFRN and SLC3A2) influence the activity of the γ-secretase complex. Down-regulation of the activity of each of the three tetraspanin web family members leads to a reduced production of amyloid β. Thus, molecules that inhibit the expression of CD81, PTGFRN and/or SLC3A2 can be used to manufacture a medicament for the treatment of Alzheimer's disease.

The nucleotide and amino acid sequence of PTGFRN (or prostaglandin F2-alpha receptor-associated protein or prostaglandin F2-alpha receptor regulatory protein or prostaglandin F2 receptor negative regulator precursor or CD315 antigen or FPRP) is, respectively, depicted in SEQ ID NOS:1 and 2.

The nucleotide and amino acid sequence of CD81 (or target of the antiproliferative antibody 1 or tetraspanin-28 or TAPA1) is, respectively, depicted in SEQ ID NOS:3 and 4. The nucleotide and amino acid sequence of SLC3A2 (or CD98 antigen or MDU1 or NACAE or 4f2 cell-surface antigen heavy chain) is, respectively, depicted in SEQ ID NOS:5 and 6.

In a particular embodiment, the molecules that inhibit the expression of PTGFRN, CD81 or SLC3A2 are short interference RNA molecules.

Thus, the invention provides the use of a short interference RNA (siRNA) hybridizing with an RNA molecule encoding a tetraspanin web family member selected from the list consisting of PTGFRN (SEQ ID NO:1), CD81 (SEQ ID NO:3) and SLC3A2 (SEQ ID NO:5) for the manufacture of a medicament to prevent and/or to treat Alzheimer's disease. The siRNA sequences are depicted in Table 2.

In another embodiment, the invention provides a pharmaceutical composition comprising an effective amount of an isolated siRNA comprising a sense RNA strand and an antisense RNA strand, wherein the sense and the antisense RNA strands form an RNA duplex, and wherein the sense RNA strand comprises a nucleotide sequence identical to a target sequence of about 19 to about 25 contiguous nucleotides in SEQ ID NOS:1 or 3 or 5.

In particular, the invention therefore provides isolated siRNA comprising short double-stranded RNA from about 19 to about 25 nucleotides in length, that are targeted to the target mRNA of SEQ ID NOS:1, 3 and/or 5. The siRNA comprise a sense RNA strand and a complementary antisense RNA strand annealed together by standard Watson-Crick base-pairing interactions (hereinafter “base-paired”). The sense strand comprises a nucleic acid sequence that is identical to a target sequence contained within the target mRNA. The sense and antisense strands of the present siRNA can comprise two complementary, single-stranded RNA molecules or can comprise a single molecule in which two complementary portions are base-paired and are covalently linked by a single-stranded “hairpin” area. The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered. The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention can also comprise a 3′ overhang. A “3′ overhang” refers to at least one unpaired nucleotide extending from the 3′-end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNAs of the invention can be targeted to any stretch of approximately 19-25 contiguous nucleotides in any of the target mRNA sequences (the “target sequence”), which sequences are depicted in SEQ ID NOS:1, 3 and 5. Techniques for selecting target sequences for siRNA are well known in the art. Thus, the sense strand of the present siRNA comprises a nucleotide sequence identical to any contiguous stretch of about 19 to about 25 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly in neurons.

The siRNAs of the invention can also be expressed from recombinant viral vectors intracellularly in neurons. The recombinant viral vectors comprise sequences encoding the siRNAs of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in the brain (e.g., in hipocampal neurons).

As used herein, an “effective amount” of the siRNA is an amount sufficient to cause RNAi-mediated degradation of the target mRNA, or an amount sufficient to inhibit the progression of plaque formation (or amyloid-β40/42 formation) in a subject. RNAi-mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA of the invention to be administered to a given subject, by taking into account factors such as the size and weight of the subject; the extent of the disease penetration; the age, health and sex of the subject; the route of administration; and whether the administration is regional or systemic. Generally, an effective amount of the siRNA of the invention comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

The present methods can be used to prevent and/or to treat plaque formation of amyloid-β in the brain of patients suffering from Alzheimer's disease. For treating Alzheimer's disease, the siRNAs of the invention (one or more siRNAs directed to one, two or three targets) can be administered to a subject in combination with a pharmaceutical agent that is different from the present siRNA. Alternatively, the siRNA of the invention can be administered to a subject in combination with another therapeutic method designed to treat Alzheimer's disease.

In the present methods, the present siRNAs (at least one or a combination of siRNAs directed against one or two or three targets) can be administered to the subject either as naked siRNA, in conjunction with a delivery reagent, or as a recombinant plasmid or viral vector that expresses the siRNA. In a particular embodiment, siRNAs are first bound to a peptide derived from Rabies virus that is coupled to a poly-Arginine stretch (YTIWMPENPRPGTPCDIFTNSRGKRASNGGGGRRRRRRRRR) (see P. Kumar et al. (2007) Nature 448 (7149):39-43) (SEQ ID NO:35). Suitable delivery reagents for administration in conjunction with the present siRNA include the Mirus Transit TKO lipophilic reagent; lipofectin; lipofectamine; cellfectin; or polycations (e.g., polylysine), or liposomes. A preferred delivery reagent is a liposome.

Liposomes can increase the blood half-life of the siRNA. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. Preferably, the liposomes encapsulating the present siRNAs comprise a ligand molecule that can target the liposome to the brain. A preferred ligand is a peptide derived from Rabies Virus (YTIWMPENPRPGTPCDIFTNSRGKRASNG) (SEQ ID NO:36) because this peptide ligand is capable of crossing the blood brain barrier and is also capable of crossing neuronal membranes.

Particularly preferably, the liposomes encapsulating the present siRNA are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the macrophage-monocyte system (“MMS”) and reticuloendothelial system (“RES”). Liposomes modified with opsonization-inhibition moieties thus remain in the circulation much longer than unmodified liposomes. For this reason, such liposomes are sometimes called “stealth” liposomes. Preferably, the opsonization-inhibiting moiety is a PEG, PPG, or derivatives thereof. Liposomes modified with PEG or PEG-derivatives are sometimes called “PEGylated liposomes.”

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. The siRNA can also be administered to a subject by gene gun, electroporation, or by other suitable parenteral or enteral administration routes. Suitable enteral administration routes include oral, rectal, or intranasal delivery. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps). In a particular embodiment, siRNAs are delivered through stereotactic injection into the brain (e.g., through intracerebroventricular injection). The siRNAs of the invention can be administered in a single dose or in multiple doses. Where the administration of the siRNAs of the invention is by infusion, the infusion can be a single sustained dose or can be delivered by multiple infusions.

One skilled in the art can also readily determine an appropriate dosage regimen for administering the siRNA (i.e., at least one siRNA) of the invention to a given subject. For example, the siRNA can be administered to the subject once, for example, as a single injection or deposition directly into the brain. Alternatively, the siRNA can be administered once or twice daily to a subject for a period of from about three to about twenty-eight days, more preferably from about seven to about ten days. Where a dosage regimen comprises multiple administrations, it is understood that the effective amount of siRNA administered to the subject can comprise the total amount of siRNA administered over the entire dosage regimen. The siRNAs of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example, as described in Remington's Pharmaceutical Science, 17th ed., Mack Publishing Company, Easton, Pa. (1985), the entire disclosure of which is herein incorporated by reference.

The present pharmaceutical formulations comprise an siRNA of the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like. Pharmaceutical compositions of the invention can also comprise conventional pharmaceutical excipients and/or additives. Suitable pharmaceutical excipients include stabilizers, antioxidants, osmolality-adjusting agents, buffers, and pH-adjusting agents. Suitable additives include physiologically biocompatible buffers (e.g., tromethamine hydrochloride), additions of chelants (such as, for example, DTPA or DTPA-bisamide) or calcium chelate complexes (as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form or can be lyophilized. For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can comprise any of the carriers and excipients listed above and 10-95%, preferably 25%-75%, of one or more siRNAs of the invention. A pharmaceutical composition for aerosol (inhalational) administration can comprise 0.01-20% by weight, preferably 1%-10% by weight, of one or more siRNAs of the invention encapsulated in a liposome as described above. A carrier can also be included as desired; e.g., lecithin for intranasal delivery.

In yet another specific embodiment, the invention uses an antibody binding to a tetraspanin web family member selected from the list consisting of PTGFRN (SEQ ID NO:2), CD81 (SEQ ID NO:4) and SLC3A2 (SEQ ID NO:6) for the manufacture of a medicament to prevent and/or to treat Alzheimer's disease.

The terms “antibody” or “antibodies” relate to an antibody characterized as being specifically directed against SEQ ID NOS:2, 4 and/or 6 or any functional derivative thereof, with the antibodies being preferably monoclonal antibodies; or an antigen-binding fragment thereof, of the F(ab′)₂, F(ab) or single chain Fv type, or any type of recombinant antibody derived thereof. These antibodies of the invention, including specific polyclonal antisera prepared against SEQ ID NOS:2, 4 and/or 6 or any functional derivative thereof, have no cross-reactivity to other proteins.

The monoclonal antibodies of the invention can, for instance, be produced by any hybridoma liable to be formed according to classical methods from splenic cells of an animal, particularly of a mouse or rat immunized against SEQ ID NOS:2, 4 and/or 6 or any functional derivative thereof, and of cells of a myeloma cell line, and to be selected by the ability of the hybridoma to produce the monoclonal antibodies recognizing SEQ ID NOS:2, 4 and/or 6, or any functional derivative thereof, which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains.

Alternatively, the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also, fragments derived from these monoclonal antibodies, such as Fab, F(ab)′₂ and scFv (“single chain variable fragment”), providing they have retained the original binding properties, form part of the present invention. Such fragments are commonly generated by, for instance, enzymatic digestion of the antibodies with papain, pepsin, or other proteases.

It is well known to the person skilled in the art that monoclonal antibodies, or fragments thereof, can be modified for various uses. The antibodies involved in the invention can be labeled by an appropriate label of the enzymatic, fluorescent, or radioactive type. In a particular embodiment, the antibodies against SEQ ID NOS:2, 4 and/or 6 or a functional fragment thereof are derived from camels. Camel antibodies are fully described in WO94/25591, WO94/04678 and in WO97/49805.

The term “medicament to treat” relates to a composition comprising molecules as described above and a pharmaceutically acceptable carrier or excipient (both terms can be used interchangeably) to prevent and/or to treat Alzheimer's disease. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers.

The “medicament” may be administered by any suitable method within the knowledge of the skilled man. One route of administration is parenterally. In parental administration, the medicament of this invention will be formulated in a unit dosage injectable form such as a solution, suspension or emulsion, in association with the pharmaceutically acceptable excipients as defined above. However, the dosage and mode of administration will depend on the individual. Generally, the medicament is administered so that the antibody of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used. If so, the medicament may be infused at a dose between 5 and 20 μg/kg/minute, more preferably between 7 and 15 μg/kg/minute.

It is clear to the person skilled in the art that the use of a therapeutic composition comprising, for example, an antibody against SEQ ID NOS:2, 4 or 6 for the manufacture of a medicament to prevent and/or to treat Alzheimer's disease can be administered by any suitable means, including, but not limited to, parenteral, subcutaneous, intraperitoneal, intrapulmonary, intracerebroventricular and intranasal administration. Parenteral infusions include intramuscular, intravenous, intra-arterial, intraperitoneal, or subcutaneous administration. In addition, the therapeutic composition is suitably administered by pulse infusion, particularly with declining doses of the antibody.

### EXAMPLES

### 1. Tandem Affinity-Tag Purification of dTag PSs and PSH1

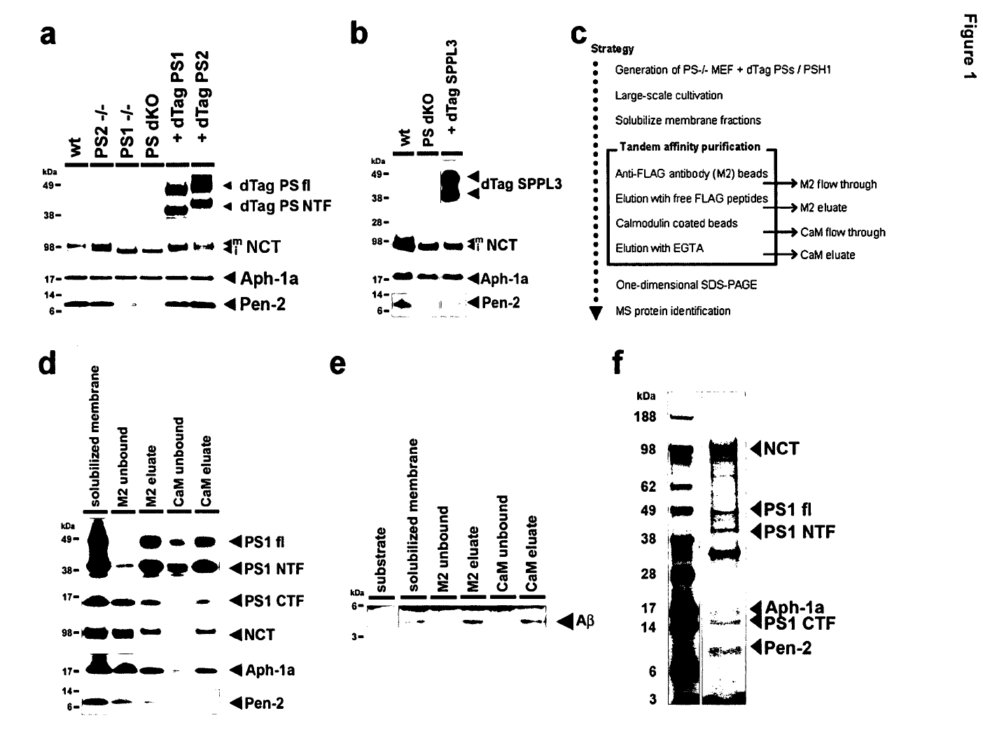

We added a double-tag (dTag) consisting of calmodulin binding protein (CBP) followed by three times FLAG sequences to the N-termini of PS1, PS2 and Presenilin homolog 1 (PSH1). PSH1, also called SPP-like protease 3 (SPPL3), is a member of the Presenilin homolog family, but has a reverse membrane topology compared to PS and is used here as a control.^{21, 22} Gamma-secretase complex maturation in PS1−/−PS2−/− (dKO) MEF was restored by stably transfecting dTag PS1 or dTag PS2, but not by dTag PSH1 (FIG. 1, Panels a and b). These complexes with dTag PS were enzymatically active as demonstrated by cleavage of APP, Notch, syndecan-3 and N-cadherin (results not shown).²³

Microsomal membranes were solubilized using CHAPSO or CHAPS and TAP was performed using anti-FLAG antibody (M2) and calmodulin (CaM) conjugated beads (summarized in FIG. 1, Panel c). Western blot analysis showed quantitative recovery of dTag-proteins (FIG. 1, Panel d). Other (endogenous) γ-secretase components, NCT, Aph-1a and Pen-2, were co-purified, although substantial amounts of these proteins appeared also in the first flow through. Since in the second binding step all components remained stable associated, we speculate that under steady state conditions, a pool of these proteins is loosely or not bound to PS. Activity of γ-secretase complex was almost completely recovered in the final sample (FIG. 1, Panel e).

We tested to what extent eluted γ-secretase could bind to immobilized transient-state analogue inhibitor WPE-III-31C, which should enrich for active γ-secretase complex.²⁴ Bound proteins were eluted with buffer containing SDS, separated on gel and visualized by Coomassie blue staining (FIG. 1, Panel f) or identified by mass spectrometric analysis. We compared protein patterns purified by three-step (with the inhibitor beads) and two-step purification using gel staining and mass spectrometric analysis, but found no significant differences. One possible interpretation is that the TAP purified γ-secretase complex is mainly in the active conformation, since only active protease is supposed to bind to this inhibitor column.²⁴ However, significant amounts of full-length PS1 were also present in the eluate, suggesting that this procedure is not entirely specific for active complexes.

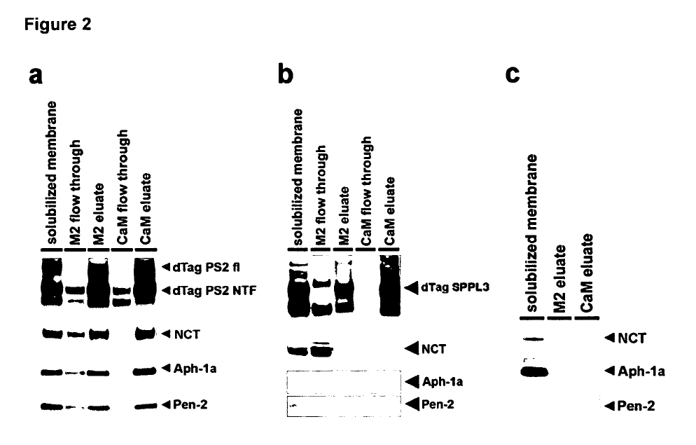

We performed purifications using dTag PS2 and obtained similar results (FIG. 2). When we purified dTag PSH1, all γ-secretase components remained in the flow-through fractions. As a final control, we also performed “mock” purification experiments with PS dKO membranes. Here also, all γ-secretase components remained in the flow-through fractions. Purification of dTag PS1 gave the largest number of bands, whereas fewer bands were detected in dTag PS2 and very few bands were visible in dTag PSH1 and PS dKO MEF purifications.

### 2. Proteomic Analysis of the PS/γ-Secretase Interacting Proteins

Coomassie blue stained bands (and the corresponding area from gels generated with PS dKO MEF) were excised and in-gel digested with trypsin and analyzed by liquid chromatography tandem mass spectrometry (LC-MS/MS). In total, five independent dTag PS1 and four dTag PS2 purifications were performed and analyzed. PS and NCT were easily identified but the number of Aph-1a and Pen-2 peptides was quite restricted. These proteins are extremely hydrophobic and all identified peptides were derived from the hydrophilic loop regions. Thus, the abundance of sequenced peptides does not necessarily correlate with the abundance of the protein in the purified material or the strength of the interaction with PS. Aph-1b and Aph-1c were not identified, likely reflecting their low expression levels in fibroblasts. After excluding proteins identified in the purification from PS dKO MEFs, which we considered as non-specific binders, we identified 59 proteins in at least two independent purifications of PS1 and PS2. Of interest, the PS2 interacting proteins selected using this criterion were also present in the PS1 interactome. Thus, PS2 and PS1 complexes interact with similar proteins in the cell. For further analysis, we did not take into account proteins that were purified with dTag PSH1, nor ribosomal and mitochondrial proteins. The fact that several of ribosomal and mitochondrial proteins were present in PS dKO and PSH1 control purifications, suggested that these proteins easily contaminate TAP purifications, although we cannot exclude the possibility that some reflect real physiological interactions.²⁵

The literature on PS1 interacting proteins is rather diffuse, but interestingly, we could confirm several previously published associations, i.e., with Tmp21/p23, alpha-catenin, β-catenin gamma-catenin and rab11.^{16, 17, 26, 27} We also identified 5-catenin, N-cadherin, ApoER2 and FKBP8, but in only one purification.²⁸⁻³¹ Other published interactions with PS were not confirmed.¹ It is possible that some of these proteins were expressed in the fibroblasts below detection limit of our assays.

### 3. Functions of PS1/γ-Secretase Interacting Proteins

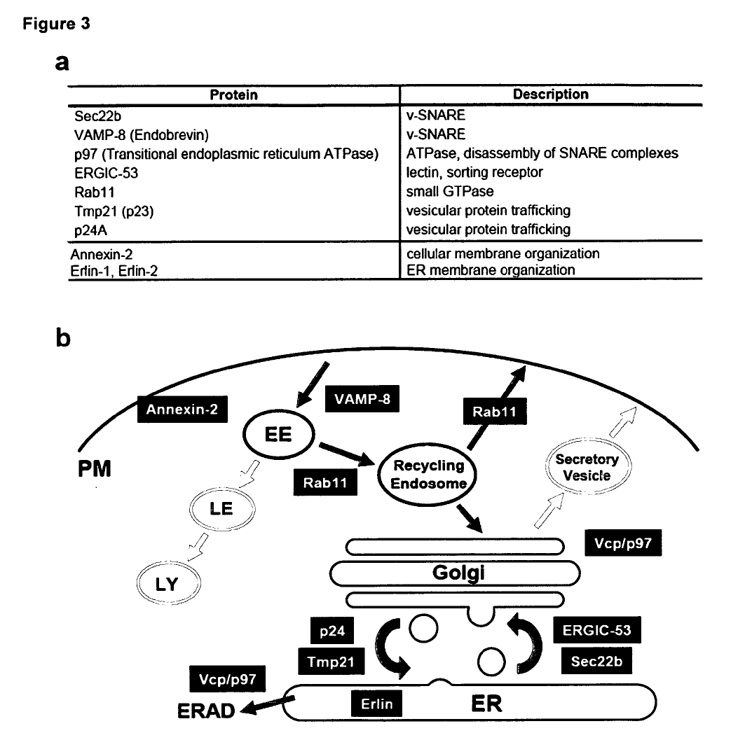

According to literature mining and gene ontology database (http://www.geneontology.org/), PS interacting proteins were assigned to various molecular functions and biological processes (Table 1). Functional subgroups, including vesicle-mediated membrane trafficking and membrane organization, indicate the importance of subcellular trafficking for the normal function of PS/γ-secretase (FIG. 3, Panel a). Sec22b is a v-SNARE involved in the docking process of ER-derived COPII coated vesicles with the cis-Golgi membrane. Members of p24 family proteins p24 and Tmp21 were proposed to serve as cargo receptors for the COPI coat protein. ERGIC-53 is a lectin that predominantly localizes in the ER-Golgi-intermediate-compartment (ERGIC). AAA-ATPase VCP/p97 serves as a molecular chaperone to disassemble SNARE complexes, but has been also implicated in the export of misfolded proteins from the ER to the cytoplasm followed by proteasomal degradation. VAMP8 is a v-SNARE protein acting at the level of endosomal sorting. Rab11 is one of the Ras-related small GTPase family proteins considered to control endosomal recycling as well as trafficking to the TGN. Furthermore Annexin-2 and Erlin have been implicated in lipid-raft-like membrane organization in plasma membrane and the ER, respectively.^{32, 33} PS resides mainly in the ER, while fully assembled complexes leave the ER and reach later compartments of the secretory pathway and the plasma membrane.

It is also assumed that an important fraction of its activity occurs in endosomal compartments.³⁴ Subcellular distribution of the γ-secretase complex can be assumed based on the localization of these membrane trafficking/organization-related proteins (FIG. 3, Panel b). The functional importance of these interactions was further confirmed using RNAi approaches and co-precipitation of endogenous proteins (see below).

Besides proteins mentioned above, a series of the identified proteins reside mainly in the ER and are involved in protein synthesis, glycosylation, or quality control, such as ribophorins, protein disulfide isomerases and heat-shock proteins. These types of proteins were also found in the analyses of PS2 or PSH1 (SPPL3), suggesting common synthetic mechanisms in the earliest membrane compartment. It is also possible that some of these chaperone proteins bind during the purification protocol, which involves some denaturation of the natural folds in the protein complex.

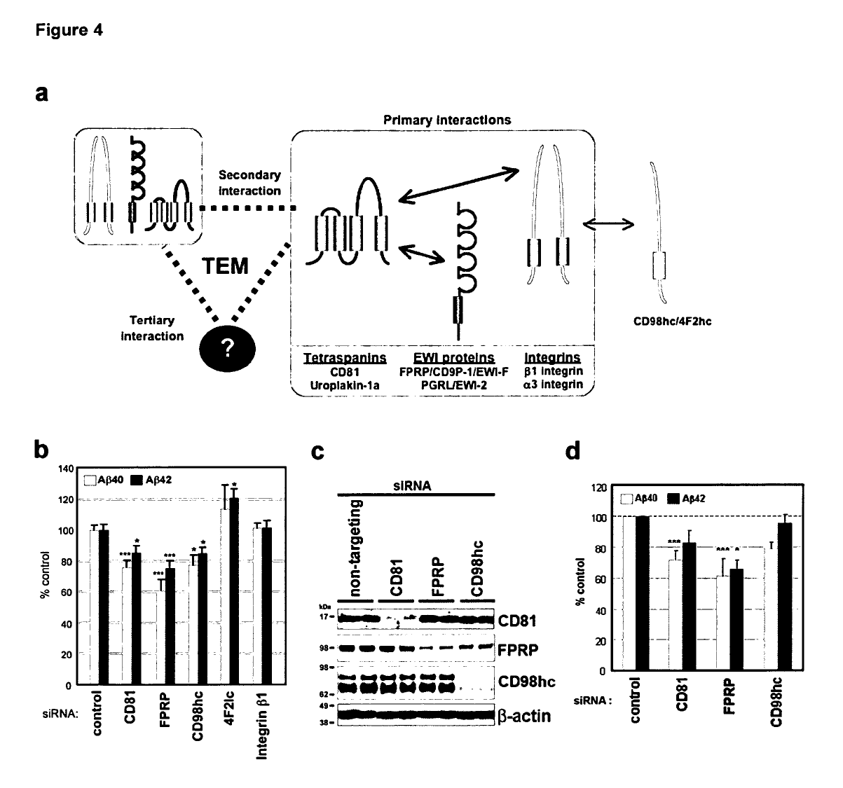

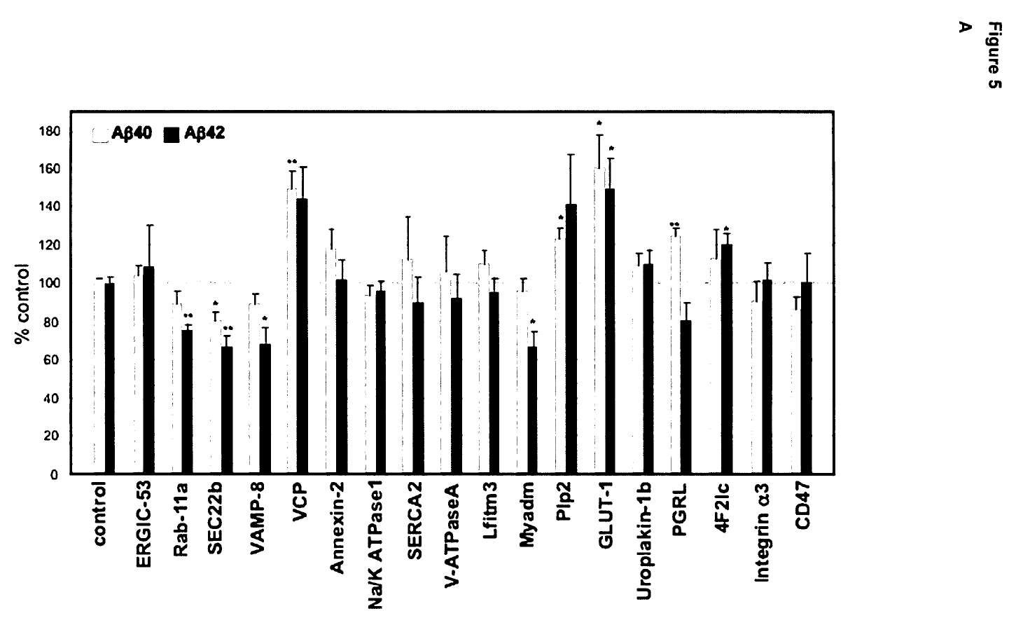

Furthermore, based on literature mining, we identified a subset of proteins that form a specific membrane microdomain called tetraspanin web and some proteins that might associate with this domain (Table 1, FIG. 4, Panel a).

### 4. Functional Interaction

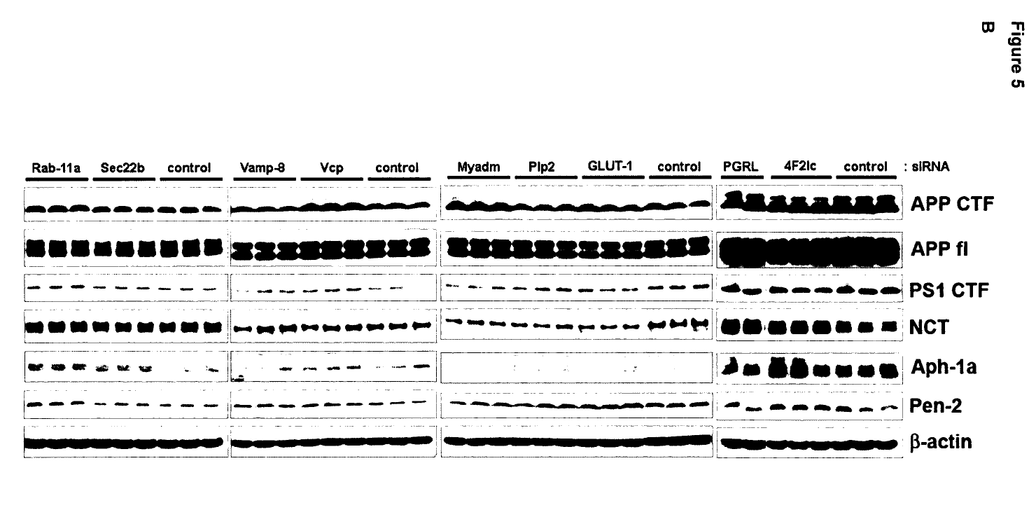

We reasoned that physiologically relevant protein-protein interactions should influence the functional properties of the complex. We, therefore, selected 24 proteins for further validation. Human embryonic kidney (HEK) 293 cells stably overexpressing the gamma-secretase substrate APPSw were transfected with siRNAs against the individual target proteins for 48 hours. Cell culture medium was collected and the γ-secretase generated Aβ40 and Aβ42 peptides were assessed using ELISA (FIG. 4b, FIG. 5). In comparison with the transfection of control (non-targeting) siRNA, eight and nine out of the 24 candidates showed statistically significant alterations of Aβ40 and Aβ42 species, respectively. Among the membrane trafficking proteins, VCP/p97 knockdown showed a significant increase of about 50% of both Aβ40 and Aβ42 secretion. On the other hand, knockdown of Sec22b, Rab11 and VAMP-8 resulted in relative mild decreases in Aβ generation. Other candidates with significant effects were Slc2a1 (GLUT-1) (˜50% increase) and the proteins involved in the tetraspanin web (FIG. 5). Knockdown of some candidates seemed to affect the levels of APP itself (see FIG. 5b, VCP/p97, Myadm or 4F2lc). It should be noticed that this initial characterization studies only further corroborate our conclusion that the identified proteins have physiological relevance for γ-secretase processing of APP but that their working mechanism needs to be further elucidated. For the interpretation of these data we also have to take into account that we did not optimize the RNAi down-regulation which is especially a problem for proteins with a long turn-over time. Therefore, our identification of proteins affecting Aβ secretion is conservative, and work in the future may reveal further functional interactions.

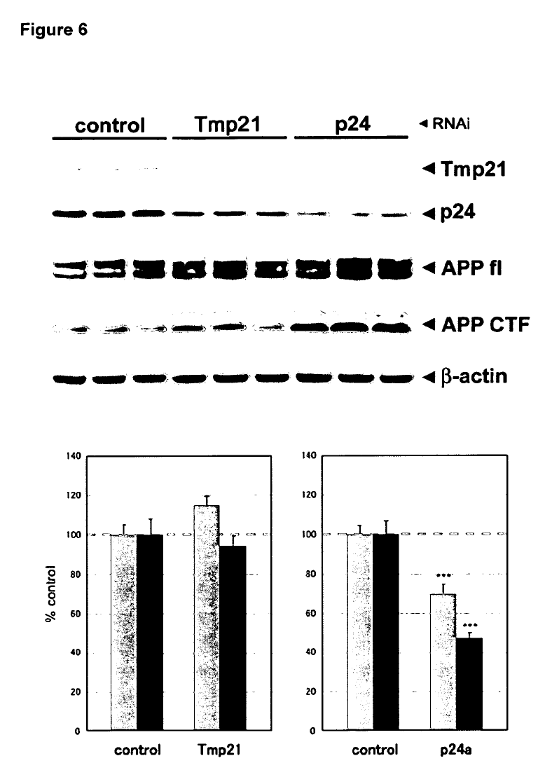

We separately examined knockdown of p24 family proteins Tmp21 and p24a as these protein family has previously been implicated in γ-secretase regulation. Tmp21 knockdown displayed slight increase in Aβ40 secretion, whereas knockdown of p24a showed opposite effect (FIG. 6).

### 5. Gamma-Secretase and the Tetraspanin Web Molecules

Tetraspanins are integral membrane proteins characterized by four transmembrane domains and conserved amino acid residues. They function as structural blocks to form a network of interactions with other tetraspanins and numerous partner (e.g. transmembrane) proteins, which are referred to as “tetraspanin webs” or “tetraspanin-enriched microdomains”^{35, 36}. Primary complexes consist of tetraspanins and direct partner proteins, such as EWI proteins and integrins α3β1 and α6β1, which assemble via homophilic lateral interactions to form higher-ordered second level complexes. They include additional interacting proteins in a third level of interaction. The tetraspanin web plays important roles in cell motility, fusion and various signalling processes^{24,25}. Tetraspanin subdomains form small raft-like microdomains in the cell membrane, endosome (etc. . . . ) and incorporate specific lipids making them floating in sucrose gradients. Core proteins of the tetraspanin web are present in the PS1 purifications (Table 1, FIG. 4a). Notably CD81, PGRL/EWI-2 (Igsf8), α3β1 integrin were identified in the PS2 interactome as well, but not in the purification with SPPL3, implying specificity of these interactions with γ-secretase.

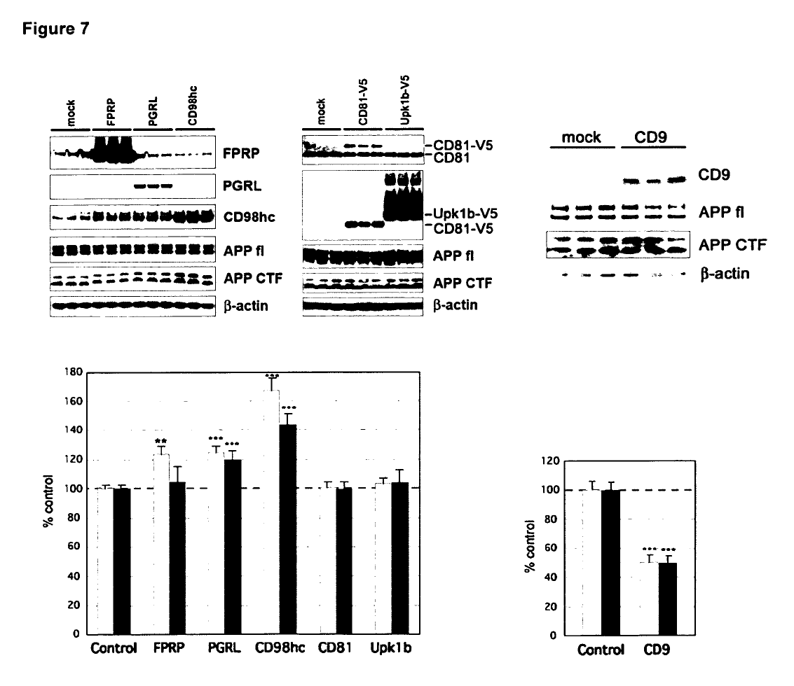

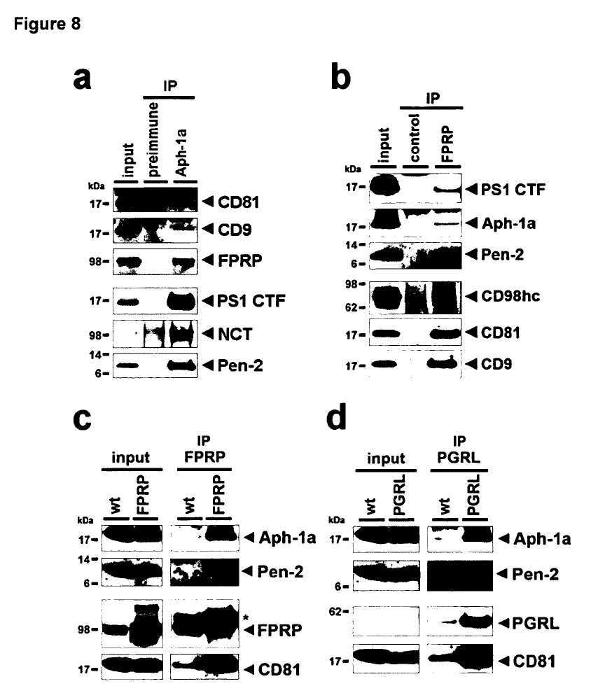

We included in our further study also the tetraspanin partner protein FPRP/CD9P-1/EWI-F (Ptgfrn), another EWI protein which is a homologue of PGRL and was identified once in the PS1 purification. Moreover peptides derived from CD98hc (SLC3A2) were frequently sequenced both in PS1 and PS2 purification. CD98hc has been implicated in regulation of the amino acid transport complex in association with CD98 light chains and in integrin signalling, indicating a possible interaction with the tetraspanin web at the level of tertiary interaction⁵¹. The primary RNAi screening in HEK293 cells indicated that knockdown of CD81, FPRP and CD98hc decreased Aβ secretion (FIG. 4b), whereas integrin RNAi did not change Aβ levels. This suggests that the former tetraspanin web components are involved in γ-secretase activity regulation, while integrins are likely only associated to γ-secretase by virtue of their incorporation into the same microdomains. We confirmed functional interaction in HeLa cells (FIGS. 4c and 4d). In both assays the knockdown effect of tetraspanin proteins on Aβ secretion was moderate (˜20-40% decrease) but significant, indicating that association of γ-secretase with the tetraspanin network is functionally relevant. Overexpression of EWI proteins and CD98hc on the other hand increased Aβ levels (FIG. 7). Interestingly expression levels of EWI proteins and CD98hc affected each other both in knockdown and overexpression experiments. This might reflect their mutual interdependence for the maintenance of the tetraspanin web. We performed co-immunoprecipitation experiments in HEK293 cells to further confirm the association of γ-secretase with the tetraspanin web. CD81 and FPRP were co-precipitated with endogenous PS1 (data not shown) and Aph-1a (FIG. 8, Panel a). Interestingly also tetraspanin protein CD9, which shares similar properties with CD81 was co-precipitated. Reciprocally, immunoprecipitation of FPRP revealed the association of endogenous γ-secretase components as well as tetraspanin partners CD81, CD9 and also CD98hc (FIG. 8, Panel b). Stable overexpression of FPRP or PGRL in HEK293 cells enhanced interaction with the γ-secretase complex (FIG. 8, Panels c and d).

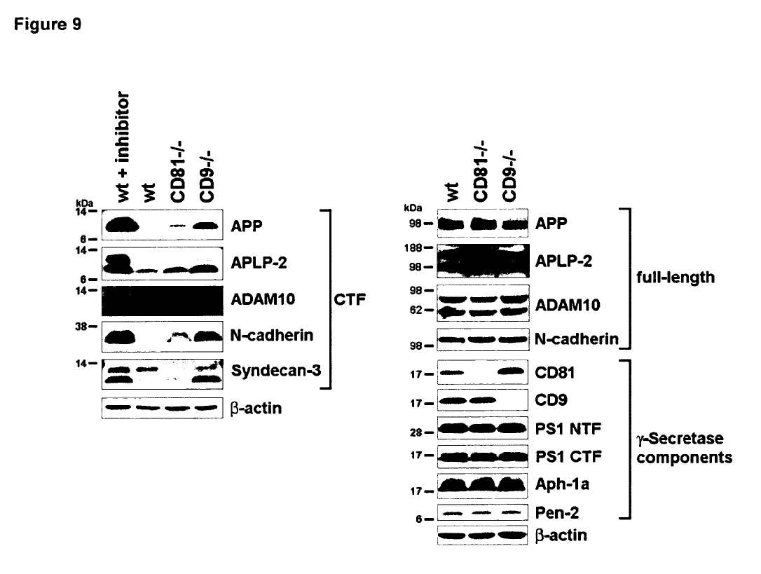

We independently confirmed the functional importance of the interaction using cells derived from CD81 or CD9 deficient mice. The carboxy-terminal fragments of endogenous γ-secretase substrates APP, APLP-2, ADAM10, N-cadherin and Syndecan-3 accumulated in those cells (FIG. 9), while expression levels of individual γ-secretase components or the substrates were unchanged. This indicated partial disruption of γ-secretase interaction with its substrates in the absence of these tetraspanin proteins.

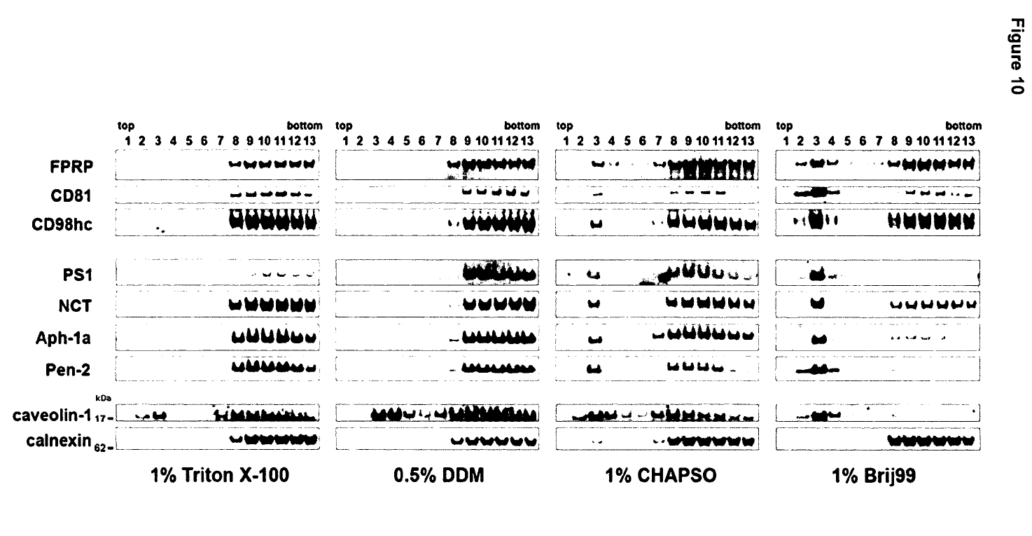

Tetraspanin proteins associate with cholesterol and gangliosides to form lipid raft-like tetraspanin-enriched microdomains (TEM), and float in sucrose density gradients using mild detergents^{42,43}. γ-Secretase also floats in detergent resistant membrane fractions^{44,45}. We examined therefore association of γ-secretase components with TEM making use of this property. HEK293 cell lysates solubilized with either Triton X-100, DDM (n-dodecyl-β-maltoside), CHAPSO or Brij99 were separated on a discontinuous sucrose gradient. As shown in FIG. 10, tetraspanin web proteins CD81, FPRP and CD98hc distributed partially together with γ-secretase components (but not the ER marker calnexin) into the low density fractions (fractions 2, 3 and 4) when membranes were solubilized with mild detergents (CHAPSO and Brij99). Caveolin, a canonical marker for cholesterol-enriched light membranes co-distributed in the top fractions as well, indicating that cholesterol and sphingolipid rich “rafts” or “caveolae” were maintained in this procedure. However, the tetraspanin web proteins, as well as the γ-secretase complex, remained in the bottom fractions upon solubilization with more stringent detergents such as Triton X-100 and DDM, while caveolin remained in the light membrane fractions. Thus, γ-secretase components co-distribute under both conditions with the fractions containing tetraspanin web proteins. These results also suggest that most, if not all γ-secretase distributing in the floating fractions is associated with those.

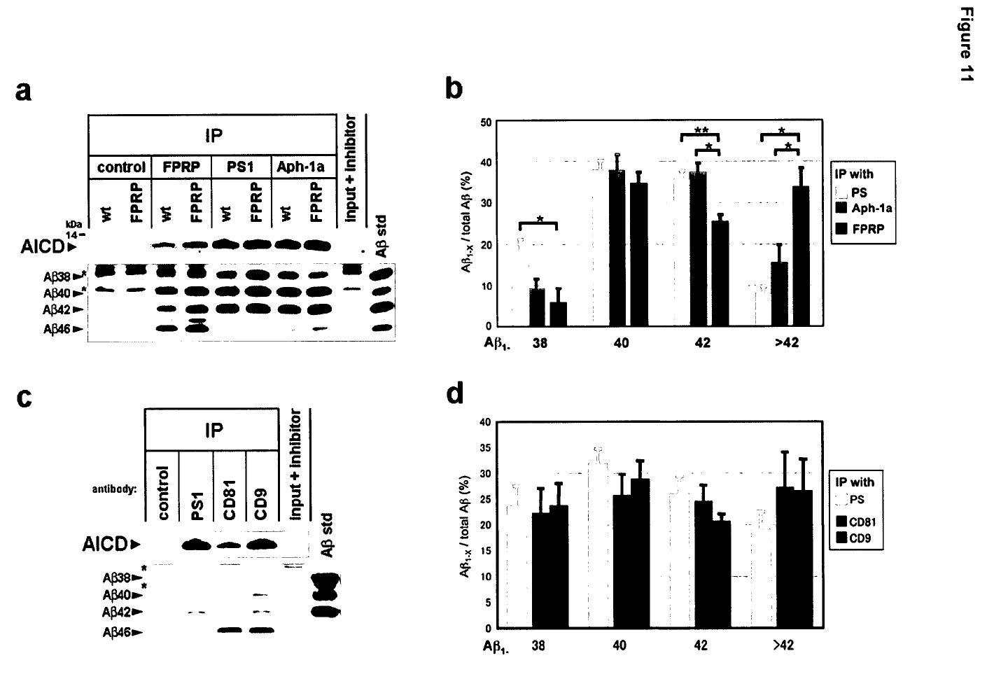

We wondered whether the association of γ-secretase with the tetraspanin domains could have a direct functional effect on its activity. We therefore immunoprecipitated γ-secretase activity associated with endogenous FPRP, CD81 or CD9 and compared its activity with the total γ-secretase activity present in cell membranes by in vitro assay. As presented by de novo generation of APP intracellular domain (AICD), active γ-secretase was co-precipitated with tetraspanin proteins (FIGS. 11a and 11c). Increased expression of FPRP in HEK293 cells augmented the co-precipitated activity, which is consistent with the result shown in FIG. 8c, while non related antibody or inhibitor controls demonstrated the specificity of the reactions. Interestingly, the spectrum of Aβ species as analyzed in urea-SDS PAGE generated by tetraspanin web-associated γ-secretase differed from the overall activity of γ-secretase pulled down with either anti-PS or Aph-1a antibodies. Tetraspanin-associated γ-secretase activity shows increased production of longer Aβ species (>Aβ₁₋₄₂), and less Aβ₁₋₃₈ and Aβ₁₋₄₂ (FIG. 11b and a table in 6c). This property of tetraspanin web-associated γ-secretase generating longer Aβ species was conserved even after 16 hours of in vitro enzyme reaction, indicating that association with the tetraspanin web rather preferably generates longer Aβ than simply delaying the successive cleavage reactions of γ-secretase. These data suggest that the different membrane or protein composition of these microdomains indeed subtly affects the activity of the γ-secretase complex.

### Materials and Methods

### Antibodies

Rabbit polyclonal anti-Aph-1A_{L} (B80.3), anti-Pen-2 (B126.2), anti-APP (B63.9) and mouse monoclonal anti-Nicastrin (9C3) have been described elsewhere.²⁵ Other antibodies were purchased: Antibodies against PS1 (MAB5232) were purchased from CHEMICON; anti-Aβ N-term (W0-2) from The Genetics Company; anti-FLAG M2 and anti-beta-actin from Sigma; anti-CD81, anti-CD9, anti-CD98hc and anti-caveolin-1 from Santa Cruz; anti-calnexin from Transduction lab. Monoclonal antibodies against FPRP (1F11) and PGRL (8A12) were kindly provided by Dr. Eric Rubinstein (Inserm, France).

### Cell Culture and Generation of the Stable Cell Lines

Immortalized mouse embryonic fibroblasts (MEF), HEK293 and HeLa cells were cultured in Dulbecco's modified Eagle's medium F-12 (Invitrogen) supplemented with 10% fetal bovine serum (Sigma). Cultures were kept at 37° C. in a humidified atmosphere containing 5% CO₂. MEF cells were generated from PS1−/−PS2−/− mouse embryos and their littermate controls.²⁵ PS dKO MEFs expressing either dTag mouse PS1, PS2 or human PSH1 were generated using a replicative-defective retrovirus system (Clontech).²⁵ Stable transfected cells were selected using 5 μg/ml puromycin (Sigma).

### Preparation of Cell Lysates and Western Blot

Total cell extracts were prepared in TBS (50 mM Tris-HCl pH 7.4, 150 mM NaCl) containing 1% Triton-X100, and Complete protease inhibitors (Roche Applied Science). Insoluble fractions were removed by centrifugation at 15,000×g for 15 minutes at 4° C. Protein concentration was determined by the Bradford dye-binding procedure (Bio-Rad). Proteins were separated on 4-12%, 10% or 12% NuPAGE Bis-Tris gels (Invitrogen) and were transferred to nitrocellulose membranes. Membranes were blocked with 5% skim milk in TBS and probed with antibodies followed by incubation with horseradish peroxidase conjugated antibodies (Bio-Rad). Bands were detected with Renaissance (ParkinElmer).

### Tandem Affinity Purification of the γ-Secretase Complex

Harvested MEF cells were re-suspended in STE buffer (5 mM Tris-HCl pH 7.4, 250 mM sucrose, 1 mM EGTA) supplemented with the Complete protease inhibitors. The cells were lysed by being passed ten times through a ball-bearing cell cracker followed by removal of the nuclei and cell debris by centrifugation at 800×g for ten minutes at 4° C. The supernatant was further centrifuged at 100,000×g for 60 minutes at 4° C. The microsomal membrane pellet was re-suspended in the solubilization buffer (HEPES buffer; 50 mM HEPES pH 7.2, 150 mM NaCl with 1% 3-[(3-Cholamidopropyl)dimethylammonio]-2-hydroxy-1-propanesulfonate (CHAPSO, Calbiochem) or 0.5% 3-[(3-Cholamidopropyl) dimethylammonio]-1-propanesulfonate (CHAPS, Calbiochem)) supplemented with the Complete protease inhibitors and was solubilized at 4° C. for three hours. After centrifugation at 100,000×g for 60 minutes at 4° C., the supernatant was saved and protein concentration was determined. The solubilized membranes were applied on pre-equilibrated anti-FLAG M2 affinity gel (Sigma). The gel was washed with the solubilization buffer and bound proteins were eluted with solubilization buffer with 20 μg/ml of FLAG peptides (Sigma). The eluate was supplemented with 2 mM CaCl₂ (final) and was applied on calmodulin-sepharose beads (Amersham Biosciences). After washing, the bound proteins were eluted with 5 mM EGTA. For further purification for dTag PS1, the eluate from calmodulin beads was applied on γ-secretase inhibitor beads, which were the agarose resin affi-gel 102 (Bio-Rad) conjugated with hydroxyethyl-urea transition state analogue inhibitor, WPE-III-31C.²⁶ After washing the beads with the solubilization buffer, the bound proteins were eluted with buffer containing 0.5% SDS.

### MS Analysis

The purified materials were concentrated and separated on 4-12% NuPAGE Bis-Tris gels. The gels were stained with GelCode Blue stain reagent (Pierce) according to the manufacturer's instructions. Protein bands were excised, in-gel digested with trypsin and following LC-MS/MS analysis as described elsewhere.⁴⁹ Proteins were identified in SwissProt or NCBI non-redundant protein database.

### Analysis of APP Processing

Twenty-four hours before transfection, HEK293 cells stably expressing APP bearing Swedish mutation (KM670/671 NL) were plated out in 24-well plates. The cells were transfected with ON-TARGETplus SMARTpool or Duplex (for Ptgfrn, Igsf8, Itgb1, Itga3, Slc3a2, CD81, CD9 and ATP1A1) siRNAs (Dharmacon) using LipofectAMINE2000 (Invitrogen). For control transfection, siCONTROL Non-targeting pool siRNA was used. Thirty-two hours after transfection, medium was changed to DMEM supplemented with 1% FBS and 16 hours later, the medium was collected. The medium was centrifuged at 800×g for five minutes at 4° C. to remove cells. Supernatant was used in a specific ELISA to detect Aβ40 and Aβ42 (The Genetics Company) according to the manufacturer's instructions. For analysis in Hela cells, cells were plated in 24-well plates and the cells were transfected with siRNAs. Twenty hours later, the cells were infected with human APP-Swedish-695 (APP695Sw) adenovirus using an infection multiplicity of 50. After six hours of infection, the cells were rinsed once with DPBS and medium was changed to DMEM supplemented with 1% FBS. Sixteen hours later, the medium was collected and subjected to ELISA.

Total cell extracts were prepared in lysis buffer (1% TritonX-100, 1% sodium deoxycholate, 0.1% SDS in HEPES buffer with Complete protease inhibitors) and insoluble fractions were removed by centrifugation at 15,000×g for 15 minutes at 4° C. Equal amounts of proteins were separated by SDS-PAGE and detected by Western blot.

For the in vitro γ-secretase assay, samples were mixed with the recombinant substrate APP C99-FLAG purified from E. Coli expressing C99-FLAG. After incubation at 37° C., de novo formed Aβ peptides were separated on 12% NuPAGE Bis-Tris gels followed by Western blot.

### Co-Immunoprecipitation

One percent CHAPSO-solubilized microsomal membranes were incubated overnight at 4° C. with the antibodies indicated in the figures. Protein G agarose beads were added and incubated for three hours. The beads were washed three times with the solubilization buffer and immunoprecipitates were eluted in NuPAGE sample buffer.

### Sucrose Density Gradient

HEK293 cells were washed twice with ice-cold PBS and solubilized with buffer-containing detergents: 1% Triton X-100, 0.5% n-dodecyl-β-maltoside (DDM), 1% CHAPSO or 1% Brij99 (Sigma) in MES buffer (25 mM MES pH6.5, 150 mM NaCl) supplemented with Complete protease inhibitors. The cells were lysed by passing through an 18-gauge needle five times and a 26-gauge needle ten times. After removing insoluble materials by centrifugation at 15,000×g for 15 minutes at 4° C., lysates were adjusted to 45% final concentration of sucrose (4.8 ml) and transferred to a centrifuge tube. A discontinuous sucrose gradient was prepared by layering 35% sucrose (4.8 ml) and 5% (2.4 ml) sucrose. Tubes were centrifuged at 38,000 rpm for 16 hours in Beckman SW41 rotor at 4° C. and 960 μl fractions were collected from the top of the gradient.

### Statistical Analysis

Data are presented as mean values and error bars indicate the standard error of the mean (SEM). The treatment groups were compared by one-way analysis of variance (ANOVA) using Dunnett's post hoc pair-wise multiple comparisons tests or two-tailed Student's t-test. Significance was set at * P<0.05; ** P<0.01; and ***P<0.001. Statistical calculations were made using the PRISM version 4 statistical software (Graph Pad Software).

### Discussion

In the present application, the interactome of PS/γ-secretase is systematically documented using the TAP approach. In the patent literature a series of proteins are disclosed that co-purify with affinity-tagged PS in human neuroblastoma cells (WO05023858). These include the known components of the complex, and also the catenin family and members of the cadherin family. However, several of the proteins we identified here are novel, and functional analysis or the association with the tetraspanin web was not disclosed in WO05/023858. A potential problem in our experiments is the use of mild detergents. These are needed to keep the γ-secretase complex intact and active, but are expected to increase the number of nonspecific interactions. We used two important negative controls, i.e. a mock purification of non-transfected cells to identify proteins that bind non-specifically to the affinity matrices, and dTag SPPL3, to identify proteins that bind to similar hydrophobic proteins as PS. We presume that some of the identified proteins interact indirectly with the complex as part of larger proteolipid structures. Such secondary and tertiary interactions are however important in a cell biological context, as we saw in the experiments with the tetraspanin web proteins. The reliability of our proteomic analyses is further confirmed by: 1) the presence of the four γ-secretase components, 2) the repeated identification of interacting proteins, 3) the overlapping patterns of PS1- and PS2-interacting proteins and the differences with the SPPL3-interactors and 4) the identification of several reported PS-interacting proteins. Although we did not further analyze the proteins identified once (in a single purification), some of those shared biological functions with the ones that we purified multiple times. The experiments with FPRP indicate that single hits could actually identify authentic PS interacting proteins. We did not identify Rer1p which is supposed to bind to either NCT or Pen-2^{37,38}. However this is expected as Rer1p dissociates once the full complex is assembled. Although our work did not identify strong regulators of γ-secretase activity, it is clear that changes in expression of several of the identified proteins can up- or down-regulate Aβ generation (FIG. 4). It should be noted that many studies in cells derived from patients suffering from genetic forms of AD have shown similar mild alterations in Aβ secretion. We suggest that the interactome we identify here provides multiple points of impact that could lead to accumulation of Aβ peptide in sporadic AD and that the identified proteins are potentially risk genes that could pop up in genome wide association studies.

We finally focused in the current study on an unexpected and novel aspect of γ-secretase, i.e. its association with a subset of proteins implicated in the formation of the tetraspanin web. Tetraspanins organize multi-molecular complexes via different levels of interactions^{35,36} which build dynamic cholesterol-containing microenvironments in the cell membrane and regulate various biological processes such as cell motility, fusion and signalling. γ-secretase components interact and co-float with tetraspanin web molecules. Altering the expression levels of tetraspanin web showed significant effects on γ-secretase dependent cleavage of various substrates. Previous work suggested that γ-secretase activity is present in buoyant detergent-resistant membranes (DRM)^{44,45}. Tetraspanin-enriched microdomains are similar but distinct from lipid rafts^{42,43}, and tetraspanins distribute not only to the plasma membrane but also to endocytic vesicles, which fits with the overall spatial distribution of active γ-secretase complex. Moreover a growing number of proteomic studies have shown that lipid rafts and TEM are different⁴⁸. Our study suggest that actually a large part of DRM associated γ-secretase is in the tetraspanin microdomains (FIG. 10).

Tetraspanins are small and hydrophobic proteins known to easily escape from mass spectrometric analysis. This probably explains why CD9, which clearly interacted with active γ-secretase in our study (FIGS. 8b and 11c), was not identified directly in our TAP analyses. We therefore speculate that other tetraspanin molecules contribute to localization of γ-secretase in the microdomains too.

Several lines of evidence suggested a role for detergent resistant microdomains in the amyloidogenic processing of APP^{44,45}. Here we demonstrate that γ-secretase in the tetraspanin web was enzymatically active and displays a change in the pattern of Aβ production, resulting in overall more long Aβ. The biological and pathological significance of such long Aβ peptides is unclear, but they remain likely associated with cells. Recent studies suggest that these long Aβ species are intermediates of successive APP CTF processing to shorter Aβ species by ε- and γ-cleavage of γ-secretase⁵² and it seems therefore that the microdomain of the tetraspanin web influences the further processing of these peptides. Also the accumulation of various γ-secretase substrates in tetraspanin deficient cells further suggest that γ-secretase complex association with the tetraspanin web is a regulation mechanism for either activity on or interaction with its various substrates. In that regard, a recent publication has demonstrated the strong dependency of γ-secretase activity with regard to the lipid composition of the membranes in which it resides⁵³. While the detailed lipid composition of the TEM has not been reported yet, tetraspanins are known to be enriched in the exosomes⁵⁴, and exosomes contain more sphingomyelins and cholesterol⁵⁵. In conclusion, our experiments provide an interactome of the γ-secretase complex and uncover the tetraspanin web as one of the subcellular sites and regulators of active γ-secretase.

TABLE 1
Identification of dTag PS interacting proteins

| | | |
|---|---|---|
| Function | Protein | Gene |

Membrane traffick and organization

| | |
|---|---|
| ERGIC-53 | Lman1 |
| Rab-11, Rab-11b | Rab11a, Rab11b |
| SEC22b | Sec22b |
| p24 | Tmed2 |
| Tmp21/p23 | Tmed10 |
| VAMP-8/Endobrevin | Vamp8 |
| p97/VCP | Vcp |
| Annexin-2 | Anxa2 |
| Erlin-1, Erlin-2 | Spfhl, Spfh2 |

Cell adhesion, signal transduction

| | |
|---|---|
| CD47/IAP | Cd47 |
| Catenin alpha-1 | Ctnna1 |
| Catenin beta-1 | Ctnnb1 |
| Integrin alpha-3 | Itga3 |
| Integrin beta-1 | Itgb1 |
| Junction plakoglobin | Jup |
| Interferon induced transmembrane protein 3 | Ifitm3 |

Tetraspanin web

| | |
|---|---|
| CD81/TAPA-1 | Cd81 |
| PGRL/EWI-2 | Igsf8 |
| Uroplakin-1b | Upk1b |

Transporter

| | | |
|---|---|---|
| Amino acid | Solute carrier family 38 | Slc38a2 |
| | CD98hc/4F2hc | Slc3a2 |
| | L-type amino acid transporter 1/4F2Ic | Slc7a5 |
| Glucose | Glucose transporter type 1 | Slc2a1 |
| Ion | Na(+)/K(+) ATPase 1 | Atp1a1 |
| | SERCA2 | Atp2a2 |
| | V-ATPase subunit d 1 | Atp6v0d1 |
| | V-ATPase subunit A | Atp6v1a |
| | VDAC-1 | Vdacl |
| | Proteolipid protein 2 | Plp2 |

Glycosylation

| | |
|---|---|
| Oligosaccharyl transferase 48 kDa subunit | Ddost |
| Ribophorin II | Rpn2 |

Protein folding

| | |
|---|---|
| Protein disulfide-isomerase | P4hb |
| Protein disulfide-isomerase A3 | Pdia3 |
| Hsc70 | Hspa8 |
| Stress-70 protein | Hspa9a |
| CPN10 /Hsp10, CPN10-like protein | Hspel , Hspe1-rs1 |
| Serpin H1/Hsp47 | Serpinh1 |

Others

| | |
|---|---|
| Aspartate aminotransferase | Got2 |
| Guanine nucleotide-binding protein G | Gnai1,2,3,Gnal,Gnao1 ,Gnat1,2 |
| Myeloid-associated differentiation marker | Myadm |
| Transmembrane protein 109/Mitsugumin-23 | Tmem109 |
| similar to MHC class I antigen | LOC547349 |

TABLE 2
sequences of siRNA-molecules
DHARMACON ON-TARGET plus Duplex

| | Catalog | Pool | | | Antisense Sequence |
|---|---|---|---|---|---|
| Gene Name | Number | Number | Accession | Sense Sequence | (with 5′ phosphate) |
| PTGFRN | J-010619-05 | | NM_020440 | AGACACACCAUCAGUAAUUUU | AAUUACUGAUGGUGUGUCUUU |
| (FPRP-1) | | | | (SEQ ID NO: 7) | (SEQ ID NO: 8) |
| PTGFRN | J-010619-06 | | NM_020440 | GUGCACAGCUCGCCUCAUGUU | CAUGAGGCGAGCUGUGCACUU |
| (FPRP-2) | | | | (SEQ ID NO: 9) | (SEQ ID NO: 10) |
| CD81 | J-017257-06 | | NM_004356 | GAACAGCUCCGUGUACUGAUU | UCAGUACACGGAGCUGUUCUU |
| | | | | (SEQ ID NO: 11) | (SEQ ID NO: 12) |
| CD9 | J-017252-05 | | NM_001769 | ACAGGAGUCUAUAUUCUGAUU | UCAGAAUAUAGACUCCUGUUU |
| | | | | (SEQ ID NO: 13) | (SEQ ID NO: 14) |
| SLC3A2 | J-003542-09 | | NM_001013251 | GGACCUUACUCCCAACUACUU | GUAGUUGGGAGUAAGGUCCUU |
| | | | | (SEQ ID NO: 15) | (SEQ ID NO: 16) |
| ITGB1 | J-004506-05 | | NM_033668 | GUGCAGAGCCUUCAAUAAAUU | UUUAUUGAAGGCUCUGCACUU |
| | | | | (SEQ ID NO: 17) | (SEQ ID NO: 18) |

DHARMACON ON-TARGET plus SMART pool

| | Catalog | Pool | | | |
|---|---|---|---|---|---|
| Gene Name | Number | Number | Accession | OLI_Sequence | |
| IGSF8 | L-015148-00 | J-015148-05 | NM_052868 | CGAAAACGGUGAUCCCUUA | (SEQ ID NO: 19) |
| | L-015148-00 | J-015148-06 | NM_052868 | GGUCAACUCUGCAGGAAGU | (SEQ ID NO: 20) |
| | L-015148-00 | J-015148-07 | NM_052868 | GUACCAAGGAUACCCAGUU | (SEQ ID NO: 21) |
| | L-015148-00 | J-015148-08 | NM_052868 | CGCCAAAGCCUAUGUUCGA | (SEQ ID NO: 22) |
| ITGA3 | L-004571-00 | J-004571-09 | NM_005501 | CCAAGGAAACCUCUAUAUU | (SEQ ID NO: 23) |
| | L-004571-00 | J-004571-10 | NM_005501 | GCGCAAGGAGUGGGACUUA | (SEQ ID NO: 24) |
| | L-004571-00 | J-004571-11 | NM_005501 | GGAGUGGCCCUACGAAGUC | (SEQ ID NO: 25) |
| | L-004571-00 | J-004571-12 | NM_005501 | GUGUACAUCUAUCACAGUA | (SEQ ID NO: 26) |
| UPK1B | L-017260-00 | J-017260-05 | NM_006952 | GAGUGCAUCUUCUUUGUAU | (SEQ ID NO: 27) |
| | L-017260-00 | J-017260-06 | NM_006952 | CGUCAAUGCUGUGUUAUGA | (SEQ ID NO: 28) |
| | L-017260-00 | J-017260-07 | NM_006952 | CAAUUGCUGUGGCGUAAAU | (SEQ ID NO: 29) |
| | L-017260-00 | J-017260-08 | NM_006952 | AAUCAGGGCUGCUAUGAAC | (SEQ ID NO: 30) |
| SLC7A5 | L-004953-01 | J-004953-09 | NM_003486 | UGAAAACUCUGGUACGAAU | (SEQ ID NO: 31) |
| | L-004953-01 | J-004953-10 | NM_003486 | GUGAACUGCUACAGCGUGA | (SEQ ID NO: 32) |
| | L-004953-01 | J-004953-11 | NM_003486 | GAGCCAACGAAGCCGGACA | (SEQ ID NO: 33) |
| | L-004953-01 | J-004953-12 | NM_003486 | GGAAGGGUGAUGUGUCCAA | (SEQ ID NO: 34) |

### REFERENCES

Parks A. L. and D. Curtis (2007). Presenilin diversifies its portfolio. Trends Genet. 23, 140-150.
2. Kaether C., C. Haass, and H. Steiner (2006). Assembly, trafficking and function of gamma-secretase. Neurodegener. Dis. 3, 275-283.
3. De Strooper B. (2003). Aph-1, Pen-2, and Nicastrin with Presenilin generate an active gamma-secretase complex. Neuron 38, 9-12.
4. De Strooper B. et al. (1998). Deficiency of presenilin-1 inhibits the normal cleavage of amyloid precursor protein. Nature 391, 387-390.
5. Wolfe M. S. et al. (1999). Two transmembrane aspartates in presenilin-1 required for presenilin endoproteolysis and gamma-secretase activity. Nature 398, 513-517.
6. Shah S. et al. (2005). Nicastrin functions as a gamma-secretase-substrate receptor. Cell 122, 435-447.
Edbauer D. et al. (2003). Reconstitution of gamma-secretase activity. Nat. Cell Biol. 5, 486-488.
8. Kimberly W. T. et al. (2003). Gamma-secretase is a membrane protein complex comprised of presenilin, nicastrin, Aph-1, and Pen-2. Proc. Natl. Acad. Sci. USA 100, 6382-6387.
9. Takasugi N. et al. (2003). The role of presenilin cofactors in the gamma-secretase complex. Nature 422, 438-441.
10. Capell A. et al. (1998). The proteolytic fragments of the Alzheimer's disease-associated presenilin-1 form heterodimers and occur as a 100-150-kDa molecular mass complex. J. Biol. Chem. 273, 3205-3211.
11. Yu G. et al. (1998). The presenilin 1 protein is a component of a high molecular weight intracellular complex that contains beta-catenin. J. Biol. Chem. 273, 16470-16475.
12. Li Y. M. et al. (2000). Presenilin 1 is linked with gamma-secretase activity in the detergent solubilized state. Proc. Natl. Acad. Sci. USA 97, 6138-6143.
13. Edbauer D., E. Winkler, C. Haass, and H. Steiner (2002). Presenilin and nicastrin regulate each other and determine amyloid beta-peptide production via complex formation. Proc. Natl. Acad. Sci. USA 99, 8666-8671.
14. Evin G. et al. (2005). Transition-state analogue gamma-secretase inhibitors stabilize a 900 kDa presenilin/nicastrin complex. Biochemistry 44, 4332-4341.
15. Zhou J. et al. (1997). Presenilin 1 interaction in the brain with a novel member of the Armadillo family. Neuroreport 8, 1489-1494.
16. Chen F. et al. (2006). TMP21 is a presenilin complex component that modulates gamma-secretase but not epsilon-gamma-secretase activity. Nature 440, 1208-1212.
17. Zhou S., H. Zhou, P. J. Walian, and B. K. Jap (2005). CD147 is a regulatory subunit of the gamma-secretase complex in Alzheimer's disease amyloid beta-peptide production. Proc. Natl. Acad. Sci. USA 102, 7499-7504.
18. Rigaut G. et al. (1999). A generic protein purification method for protein complex characterization and proteome exploration. Nat. Biotechnol. 17, 1030-1032.
19. Gavin A. C. et al. (2002). Functional organization of the yeast proteome by systematic analysis of protein complexes. Nature 415, 141-147.
20. Bouwmeester T. et al. (2004). A physical and functional map of the human
TNF-alpha/NF-kappa B signal transduction pathway. Nat. Cell Biol. 6, 97-105.
21. Ponting C. P. et al. (2002). Identification of a novel family of presenilin homologues. Hum. Mol. Genet. 11, 1037-1044.
22. Weihofen A., K. Binns, M. K. Lemberg, K. Ashman, and B. Martoglio (2002). Identification of signal peptide peptidase, a presenilin-type aspartic protease. Science 296, 2215-2218.
23. Bentahir M. et al. (2006). Presenilin clinical mutations can affect gamma-secretase activity by different mechanisms. J. Neurochem. 96, 732-742.
24. Esler W. P. et al. (2002). Activity-dependent isolation of the presenilin-gamma-secretase complex reveals nicastrin and a gamma substrate. Proc. Natl. Acad. Sci. USA 99, 2720-2725.
25. Hansson C. A. et al. (2004). Nicastrin, presenilin, APH-1, and PEN-2 form active gamma-secretase complexes in mitochondria. J. Biol. Chem. 279, 51654-51660.
26. Raurell I., J. Castano, C. Franci, A. Garcia de Herreros, and M. Dunach (2006). Presenilin-1 interacts with plakoglobin and enhances plakoglobin-Tcf-4 association. Implications for the regulation of beta-catenin/Tcf-4-dependent transcription. J. Biol. Chem. 281, 1401-1411.
27. Dumanchin C. et al. (1999). Presenilins interact with Rab11, a small GTPase involved in the regulation of vesicular transport. Hum. Mol. Genet. 8, 1263-1269.
28. Levesque G. et al. (1999). Presenilins interact with armadillo proteins including neural-specific plakophilin-related protein and beta-catenin. J. Neurochem. 72, 999-1008.
29. Georgakopoulos A. et al. (2000). Presenilin-1: a component of synaptic and endothelial adherens junctions. Ann. N.Y. Acad. Sci. 920, 209-214.
30. May P., H. H. Bock, J. Nimpf, and J. Herz. Differential glycosylation regulates processing of lipoprotein receptors by gamma-secretase. J. Biol. Chem. 278, 37386-37392.
31. Wang H. Q. et al. (2005). Interaction of presenilins with FKBP38 promotes apoptosis by reducing mitochondrial Bcl-2. Hum. Mol. Genet. 14, 1889-1902.
32. Harder T. and V. Gerke (1994). The annexin II2p11(2) complex is the major protein component of the triton X-100-insoluble low-density fraction prepared from MDCK cells in the presence of Ca2+. Biochim. Biophys. Acta 1223, 375-382.
33. Browman D. T., M. E. Resek, L. D. Zajchowski, and S. M. Robbins (2006). Erlin-1 and erlin-2 are novel members of the prohibitin family of proteins that define lipid-raft-like domains of the ER. J. Cell Sci. 119, 3149-3160.
34. Annaert W. and B. De Strooper (1999). Presenilins: molecular switches between proteolysis and signal transduction. Trends Neurosci. 22, 439-443.
35. Boucheix C. and E. Rubinstein (2001). Tetraspanins. Cell. Mol. Life Sci. 58, 1189-1205.
36. Hemler M. E. (2005). Tetraspanin functions and associated microdomains. Nat. Rev. Mol. Cell. Biol. 6, 801-811.
37. Kaether C. et al. (2007). Endoplasmic reticulum retention of the gamma-secretase complex component Pen2 by Rer1. EMBO Rep. 8, 743-748.
38. Spasic D. et al. (2007). Rer1p competes with APH-1 for binding to nicastrin and regulates gamma-secretase complex assembly in the early secretory pathway. J. Cell Biol. 176, 629-640.
39. Vetrivel K. S. et al. (2007). Dual roles of the transmembrane protein p23/TMP21 in the modulation of amyloid precursor protein metabolism. Mol. Neurodegener. 2:4.
40. Thinakaran G. and A. T. Parent (2004). Identification of the role of presenilins beyond Alzheimer's disease. Pharmacol. Res. 50, 411-418.
41. Morais V. A. et al. (2006). N-glycosylation of human nicastrin is required for interaction with the lectins from the secretory pathway calnexin and ERGIC-53. Biochim. Biophys. Acta 1762, 802-810.
42. Charrin S. et al. (2003). Multiple levels of interactions within the tetraspanin web. Biochem. Biophys. Res. Commun. 304, 107-112.
43. Claas C., C. S. Stipp, and M. E. Hemler (2001). Evaluation of prototype transmembrane 4 superfamily protein complexes and their relation to lipid rafts. J. Biol. Chem. 276, 7974-7984.
44. Wahrle S. et al. (2002). Cholesterol-dependent gamma-secretase activity in buoyant cholesterol-rich membrane microdomains. Neurobiol. Dis. 9, 11-23.
45. Ehehalt R., P. Keller, C. Haass, C. Thiele, and K. Simons (2003). Amyloidogenic processing of the Alzheimer beta-amyloid precursor protein depends on lipid rafts. J. Cell Biol. 160, 113-123.
46. Andre M. et al. (2006). Proteomic analysis of the tetraspanin web using LC-ESI-MS/MS and MALDI-FTICR-MS. Proteomics 6, 1437-1449.
47. Le Naour F. et al. (2006). Profiling of the tetraspanin web of human colon cancer cells. Mol. Cell. Proteomics 5, 845-857.
48. Le Naour F., M. Andre, C. Boucheix, and E. Rubinstein (2006). Membrane microdomains and proteomics: lessons from tetraspanin microdomains and comparison with lipid rafts. Proteomics 6, 6447-6454.
49. Lavens D. et al. (2006). A complex interaction pattern of CIS and SOCS2 with the leptin receptor. J. Cell Sci. 119, 2214-2224.
50. Kolesnikova T. V., B. A. Mannion, F. Berditchevski, and M. E. Hemler (2001). Betel integrins show specific association with CD98 protein in low density membranes. BMC Biochem. 2:10.
51. Fenczik, C. A., Sethi, T., Ramos, J. W., Hughes, P. E. & Ginsberg, M. H. Complementation of dominant suppression implicates CD98 in integrin activation. Nature 390, 81-85 (1997).
52. Qi-Takahara, Y. et al. Longer forms of amyloid beta protein: implications for the mechanism of intramembrane cleavage by gamma-secretase. J. Neurosci. 25, 436-445 (2005).
53. Osenkowski P, Ye W, Wang R, Wolfe M S, Selkoe D J. Direct and potent regulation of gamma-secretase by its lipid microenvironment. J Biol. Chem. 2008; 283(33):22529-40.
54. Escola J M, Kleijmeer M J, Stoorvogel W, Griffith J M, Yoshie O, Geuze H J. Selective enrichment of tetraspan proteins on the internal vesicles of multivesicular endosomes and on exosomes secreted by human B-lymphocytes. J Biol. Chem. 1998; 273(32):20121-7.
55. Subra C, Laulagnier K, Perret B, Record M. Exosome lipidomics unravels lipid sorting at the level of multivesicular bodies. Biochimie. 2007; 89(2):205-12.

1. A method of treating and/or preventing Alzheimer's disease in a subject, the method comprising:
utilizing an antibody binding to a peptide selected from the group consisting of SEQ ID NO:2, SEQ ID NO:4, and SEQ ID NO:6,
so as to prevent and/or to treat Alzheimer's disease in the subject.

2. A method of treating and/or preventing Alzheimer's disease in a subject, the method comprising:
hybridizing a short interference RNA (siRNA) with an RNA molecule encoding a tetraspanin web family member selected from the group consisting of SEQ ID NO:1, SEQ ID NO:3, and SEQ ID NO:5.,
so as to prevent and/or to treat Alzheimer's disease in the subject.

3. A composition comprising:
an effective amount of an isolated short interference RNA (siRNA) comprising a sense RNA strand and an antisense RNA strand to hybridize with an RNA molecule encoding a tetraspanin web family member selected from the group consisting of PTGFRN, CD81, and/or SLC3A2,
wherein the sense and the antisense RNA strands form an RNA duplex, and
wherein the sense RNA strand comprises a nucleotide sequence identical to a target sequence of about 19 to about 25 contiguous nucleotides in SEQ ID NO: 1, 3 and/or 5 respectively.

4. The method according to claim 1, wherein the antibody is directed against SEQ ID NO:2.

5. The method according to claim 1, wherein the antibody is directed against SEQ ID NO:4.

6. The method according to claim 1, wherein the antibody is directed against SEQ ID NO:6.

7. The method according to claim 2, wherein the tetraspanin web family member is SEQ ID NO:1.

8. The method according to claim 2, wherein the tetraspanin web family member is SEQ ID NO:3.

9. The method according to claim 2, wherein the tetraspanin web family member is SEQ ID NO:5.

10. The composition of claim 3, wherein the target sequence is from about 19 to about 25 contiguous nucleotides of SEQ ID NO:1.

11. The composition of claim 3, wherein the target sequence is from about 19 to about 25 contiguous nucleotides of SEQ ID NO:3.

12. The composition of claim 3, wherein the target sequence is from about 19 to about 25 contiguous nucleotides of SEQ ID NO:5.


SEQUENCE LISTING (36 sequences)

SEQ ID NO: 1 - DNA, 6160 bp, Homo sapiens
ctcgcgaggagagcggagcaggcgcgcggcccaggcggaggagcgccgactctggagcagccggagctggaagaggaggaggaggagaggcggcggggaaggaggaggagggggagagtcgctcccgccgggcgagcatggggcgcctggcctcgaggccgctgctgctggcgctcctgtcgttggctctttgccgagggcgtgtggtgagagtccccacagcgaccctggttcgagtggtgggcactgagctggtcatcccctgcaacgtcagtgactatgatggccccagcgagcaaaactttgactggagcttctcatctttggggagcagctttgtggagcttgcaagcacctgggaggtggggttcccagcccagctgtaccaggagcggctgcagaggggcgagatcctgttaaggcggactgccaacgacgccgtggagctccacataaagaacgtccagccttcagaccaaggccactacaaatgttcaacccccagcacagatgccactgtccagggaaactatgaggacacagtgcaggttaaagtgctggccgactccctgcacgtgggccccagcgcgcggcccccgccgagcctgagcctgcgggagggggagcccttcgagctgcgctgcactgccgcctccgcctcgccgctgcacacgcacctggcgctgctgtgggaggtgcaccgcggcccggccaggcggagcgtcctcgccctgacccacgagggcaggttccacccgggcctggggtacgagcagcgctaccacagtggggacgtgcgcctcgacaccgtgggcagcgacgcctaccgcctctcagtgtcccgggctctgtctgccgaccagggctcctacaggtgtatcgtcagcgagtggatcgccgagcagggcaactggcaggaaatccaagaaaaggccgtggaagttgccaccgtggtgatccagccatcagttctgcgagcagctgtgcccaagaatgtgtctgtggctgaaggaaaggaactggacctgacctgtaacatcacaacagaccgagccgatgacgtccggcccgaggtgacgtggtccttcagcaggatgcctgacagcaccctacctggctcccgcgtgttggcgcggcttgaccgtgattccctggtgcacagctcgcctcatgttgctttgagtcatgtggatgcacgctcctaccatttactggttcgggatgttagcaaagaaaactctggctactattactgccacgtgtccctgtgggcacccggacacaacaggagctggcacaaagtggcagaggccgtgtcttccccagctggtgtgggtgtgacctggctagaaccagactaccaggtgtacctgaatgcttccaaggtccccgggtttgcggatgaccccacagagctggcatgccgggtggtggacacgaagagtggggaggcgaatgtccgattcacggtttcgtggtactacaggatgaaccggcgcagcgacaatgtggtgaccagcgagctgcttgcagtcatggacggggactggacgctaaaatatggagagaggagcaagcagcgggcccaggatggagactttattttttctaaggaacatacagacacgttcaatttccggatccaaaggactacagaggaagacagaggcaattattactgtgttgtgtctgcctggaccaaacagcggaacaacagctgggtgaaaagcaaggatgtcttctccaagcctgttaacatattttgggcattagaagattccgtgcttgtggtgaaggcgaggcagccaaagcctttctttgctgccggaaatacatttgagatgacttgcaaagtatcttccaagaatattaagtcgccacgctactctgttctcatcatggctgagaagcctgtcggcgacctctccagtcccaatgaaacgaagtacatcatctctctggaccaggattctgtggtgaagctggagaattggacagatgcatcacgggtggatggcgttgttttagaaaaagtgcaggaggatgagttccgctatcgaatgtaccagactcaggtctcagacgcagggctgtaccgctgcatggtgacagcctggtctcctgtcaggggcagcctttggcgagaagcagcaaccagtctctccaatcctattgagatagacttccaaacctcaggtcctatatttaatgcttctgtgcattcagacacaccatcagtaattcggggagatctgatcaaattgttctgtatcatcactgtcgagggagcagcactggatccagatgacatggcctttgatgtgtcctggtttgcggtgcactcttttggcctggacaaggctcctgtgctcctgtcttccctggatcggaagggcatcgtgaccacctcccggagggactggaagagcgacctcagcctggagcgcgtgagtgtgctggaattcttgctgcaagtgcatggctccgaggaccaggactttggcaactactactgttccgtgactccatgggtgaagtcaccaacaggttcctggcagaaggaggcagagatccactccaagcccgtttttataactgtgaagatggatgtgctgaacgccttcaagtatcccttgctgatcggcgtcggtctgtccacggtcatcgggctcctgtcctgtctcatcgggtactgcagctcccactggtgttgtaagaaggaggttcaggagacacggcgcgagcgccgcaggctcatgtcgatggagatggactaggctggcccgggaggggagtgacagagggacgttctaggagcaattggggcaagaagaggacagtgatattttaaaacaaagtgtgttacactaaaaaccagtcctctctaatctcaggtgggacttggcgctctctcttttctgcatgtcaagttctgagcgcggacatgtttaccagcacacggctcttcttcccacggcactttctgatgtaacaatcgagtgtgtgttttcccaactgcagctttttaatggttaaccttcatctaattttttttctcccactggtttatagatcctctgacttgtgtgtgtttatagcttttgtttcgcggggttgtggtgaggaaggggtgatggcatgcggagttctttatcttcagtgagaatgtgcctgcccgcctgagagccagcttccgcgttggaggcacgtgttcagagagctgctgagcgccaccctctacccggctgacagacaacacagacctgtgccgaaggctaatttgtggcttttacgaccctaccccaccccctgttttcaggggtttagactacatttgaaatccaaacttggagtatataacttcttattgagcccaactgctttttttttttttttttttgcttctctgccccttttccatttcttttgtatttgttttctgtgagagcactgaaatggcagccctggaatctacaatttggctctccactgagcaccttatcttgccaccttagccttaagaatgaatatgaagaaaaatacacagccacctctgtccagggcagtaagaagggctgcaaggaaggggaggatggggacaaggaaaggatcagatacctgctccagtagttgtgaggccactgtgtctcaggggactccaggaggagcagaagagggatcccacgaagttattcttacgcagctggggccaggagggtcagagtggtgccaggtgcaagttaggctaaagaagccaccactattcctctctcttgcccattgtggggggcaaaggcattggtcaccaagagtcttgcagggggacccacagatatgccatgtccttcacacgtgcttgggctccttaacctgaaggcaaattgctacttgcaagactgactgacttcaaggaatcagaaattacctagaagcaccatgttttttctatgaccttttcagtccttcaggtcattttaaggtccactgcagggggttagtgagaaagggtatactttgtggtatgttttgctttcctaatagggacatgaaggaaacccagcaatttgctgttatgtgaatggcctgtagagcagagtcaagagcggtgtgctttgcccgactgctcccatcaggaataggagagtagacagagatcttccacatcccaggcttctgctgctgctttaaaagctctgtccttggagcctcccgctccctgaagtgtctcgccccctgcacagcactggcctttcggaagcatcccagtagggttttctgaggctcgctggtgactcatgccctaattgcaatcctctgcttttatcttgactttgaaggatctaacactgctctctcttccaaaggggaaaaaaagattcatttgttttgagcaataaactaatacaaaatgatggccattcatgtgcagctctttgtcaccatgggccggatgagttgtgctcctcctggctcaccatttccccctgctcccccacagccggttctgcacttatcaccgagtcgcccctggaagcagattcccattgagttttccccaccaaggggaccatgcacatggtagaaacattagattctgcattgacagtagcctttccttggcccgggcctgtggtgggaagacgggcaacaagtataccccaccagggcctgagtgactagaggaagaggacgaggccttgttggcactagatttgggtattttctgcatgtcataacatatcctaactgctatttcagaagaggcagcttgtaggtgattgtacaagtgagaattaaagagagaacagatatttaaacaggtgctgtattagtaacagccagtgccctttcagcccttgcatctattaaaaggagattcaggattttattggcacaggcccttcttagtaggaagaaagggtgcttagctttggacctgaccgggtgtgtgtaaaaccatggactgagtcacagcagacactcgatggtggtaaatgtgatgggtgcttacacactgtaccttttcctttcatactgatgctgcagttcagggctggagttgttaaggcattgacctccacccacctgccccatgtccactgggctgcccaagctgcatgtcacctgagggctggcaggaaggggcgagaaatcccagggcattgtaccaaggacctagttccttctagggatataaatttccaggaatgtgtatttttaatgtggtgagatgcactcttttgttgtaccaaatagggctccccaccccacccctgcgacaagtgctcttctagaacaggttcctaccagcagcactggtgtgaatgaaagagagacccagccgcgtctcacacaggtggaattgcacttcttaacaaaaaggaactttataaaagtttgggattttttttcctaatcataaaaatagccccagaaagagcctaagctatgttcagatagaagcctcgaaattcctgtaaattgtttactttatgatgtttacatacacgtttcactttgaaaaaaaatgcaaatcgactttttaacaactgttgagatgtttcatgggacagtagaactctgactcaccaactgggctaaattttaatttaaaaatgtatttatttgagtgtctttccccccctcaccctcaccatctgaggggctccctgagatcttggtagaggaggcccctcctgcccagaccttcgtttgtttccccggtggcccttgcttcttgctttgcagactgcctgcagccatgattttgtcactgacatctgtgagccaaagactgagcctttttggcaggaataataagcaatactacacaacttgctactttcagaaaacttttttttagcttcaccgatgacaacagaggaagaagggaactgggatttgggtaagttctcctccactgtttgaccaaattctcagtgataaatatgtgtgcagatccctagaagagaaaacgctgactttctttttaagtgtggcacataaggatctgcagaattttccgtagacaaagaaaggatcttgtgtatttttgtccatatccaatgttatatgaactaattgtattgttttatactgtgaccacaaatattatgcaatgcaccatttgttttttatttcattaaaggaagtttaattt

SEQ ID NO: 2 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - DNA, 711 bp, Homo sapiens
atgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactag

SEQ ID NO: 4 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 5 - DNA, 2408 bp, Homo sapiens
cctgaacttgcggcgcgaaaaaggcgcgcatgcgtcctacgggagcgtgctggctcaccgaccgcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcaaaaaaaaaaa

SEQ ID NO: 6 - protein, 661 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 7 - DNA, 21 bp, Artificial Sequence
  siRNA molecule sense FPRP-1
agacacaccaucaguaauuuu

SEQ ID NO: 8 - DNA, 21 bp, Artificial Sequence
  siRNA molecule antisense FPRP-1
aauuacugauggugugucuuu

SEQ ID NO: 9 - DNA, 21 bp, Artificial Sequence
  siRNA molecule sense FPRP-2
gugcacagcucgccucauguu

SEQ ID NO: 10 - DNA, 21 bp, Artificial Sequence
  siRNA molecule antisense FPRP-2
caugaggcgagcugugcacuu

SEQ ID NO: 11 - DNA, 21 bp, Artificial Sequence
  siRNA molecule antisense CD81
gaacagcuccguguacugauu

SEQ ID NO: 12 - DNA, 21 bp, Artificial Sequence
  siRNA molecule antisense CD81
ucaguacacggagcuguucuu

SEQ ID NO: 13 - DNA, 21 bp, Artificial Sequence
  siRNA molecule sense CD9
acaggagucuauauucugauu

SEQ ID NO: 14 - DNA, 21 bp, Artificial Sequence
  siRNA molecule antisense CD9
ucagaauauagacuccuguuu

SEQ ID NO: 15 - DNA, 21 bp, Artificial Sequence
  siRNA molecule sense SLC3A2
ggaccuuacucccaacuacuu

SEQ ID NO: 16 - DNA, 21 bp, Artificial Sequence
  siRNA molecule antisense SLC3A2
guaguugggaguaagguccuu

SEQ ID NO: 17 - DNA, 21 bp, Artificial Sequence
  siRNA molecule sense ITGB1
gugcagagccuucaauaaauu

SEQ ID NO: 18 - DNA, 21 bp, Artificial Sequence
  siRNA molecule antisense ITGB1
uuuauugaaggcucugcacuu

SEQ ID NO: 19 - DNA, 19 bp, Artificial Sequence
  siRNA molecule IGSF8
cgaaaacggugaucccuua

SEQ ID NO: 20 - DNA, 19 bp, Artificial Sequence
  siRNA molecule IGFS8
ggucaacucugcaggaagu

SEQ ID NO: 21 - DNA, 19 bp, Artificial Sequence
  siRNA molecule IGSF8
guaccaaggauacccaguu

SEQ ID NO: 22 - DNA, 19 bp, Artificial Sequence
  siRNA molecule IGSF8
cgccaaagccuauguucga

SEQ ID NO: 23 - DNA, 19 bp, Artificial Sequence
  siRNA molecule ITGA3
ccaaggaaaccucuauauu

SEQ ID NO: 24 - DNA, 19 bp, Artificial Sequence
  siRNA molecule ITGA3
gcgcaaggagugggacuua

SEQ ID NO: 25 - DNA, 19 bp, Artificial Sequence
  siRNA molecule ITGA3
ggaguggcccuacgaaguc

SEQ ID NO: 26 - DNA, 19 bp, Artificial Sequence
  siRNA molecule ITGA3
guguacaucuaucacagua

SEQ ID NO: 27 - DNA, 19 bp, Artificial Sequence
  siRNA molecule UPK1B
gagugcaucuucuuuguau

SEQ ID NO: 28 - DNA, 19 bp, Artificial Sequence
  siRNA molecule UPK1B
cgucaaugcuguguuauga

SEQ ID NO: 29 - DNA, 19 bp, Artificial Sequence
  siRNA molecule UPK1B
caauugcuguggcguaaau

SEQ ID NO: 30 - DNA, 19 bp, Artificial Sequence
  siRNA molecule UPK1B
aaucagggcugcuaugaac

SEQ ID NO: 31 - DNA, 19 bp, Artificial Sequence
  siRNA molecule SLC7A5
ugaaaacucugguacgaau

SEQ ID NO: 32 - DNA, 19 bp, Artificial Sequence
  siRNA molecule SLC7A5
gugaacugcuacagcguga

SEQ ID NO: 33 - DNA, 19 bp, Artificial Sequence
  siRNA molecule SLC7A5
gagccaacgaagccggaca

SEQ ID NO: 34 - DNA, 19 bp, Artificial Sequence
  siRNA molecule SLC7A5
ggaagggugauguguccaa

SEQ ID NO: 35 - protein, 41 aa, Artificial Sequence
  Peptide derived from Rabies virus that is coupled to a poly Arginine stretch
YTIWMPENPRPGTPCDIFTNSRGKRASNGGGGRRRRRRRRR

SEQ ID NO: 36 - protein, 29 aa, Artificial Sequence
  Peptide derived from Rabies Virus
YTIWMPENPRPGTPCDIFTNSRGKRASNG